(19) United States
(12) Patent Application Publication
(10) Pub. No.: US 2023/0285601 A1
(43) Pub. Date: Sep. 14, 2023

(54) NON DEGRADABLE RADIO-OPAQUE EMBOLISATION MICROSPHERE

(71) Applicant: GUERBET, Villepinte (FR)

(72) Inventors: Anne BEILVERT, MASSY (FR); Emeline CORUS, JANVRY (FR); Laurent BEDOUET, LE MANS (FR); Olivier FOUGERE, MORTEFONTAINE (FR)

(21) Appl. No.: 17/754,532

(22) PCT Filed: Oct. 7, 2020
(86) PCT No.: PCT/EP2020/078180

§ 371 Date: Apr. 5, 2022

(30) Foreign Application Priority Data
Oct. 7, 2019 (FR) 19306304.7
Oct. 7, 2019 (FR) 19306307.0

(51) Int. Cl. A61K 49/04; A61K 49/18

(52) U.S. Cl. CPC A61K 49/0442; A61K 49/048; A61K 49/1854

(57) ABSTRACT

The invention relates to a polymer comprising a crosslinked matrix, the matrix being based on at least: a) 20 to 90% hydrophilic monomer; b) 5 to 50% radio-opaque halogenated monomer; c) 1 to 15% non-biodegradable hydrophilic crosslinking agent; and d) 0.1 to 10% transfer agent chosen among the alkyl halides and cycloaliphatic or aliphatic thiols having, in particular, 2 to 24 carbon atoms, and optionally having another functional group chosen among the amino, hydroxy and carboxy groups. The invention further relates to a pharmaceutical composition comprising at least one polymer according to the invention, in association with a pharmaceutically acceptable vehicle, advantageously for a parenteral administration. The invention further relates to a kit comprising a pharmaceutical composition comprising the polymer according to the invention in association with a pharmaceutically acceptable vehicle for a parenteral administration, and an injection means.

### FIELD OF THE INVENTION

The present invention relates to a nonbiodegradable radiopaque polymer, in particular suitable for being implanted in an individual and optionally for controlled release of active ingredients or macromolecules. The nonbiodegradable radiopaque polymer according to the invention forms in particular nonbiodegradable radiopaque embolization microspheres intended to be injected in an individual. The present invention also relates to a pharmaceutical composition comprising the polymer according to the invention.

### PRIOR ART

Therapeutic vascular occlusion (i.e. embolization) is used for preventing or treating certain pathological conditions in situ. It can be carried out by means of catheters making it possible, under imaging control, to position particulate occlusion agents (i.e. emboli or embolic agents) in the circulatory system. It has a variety of medical applications such as the treatment of vascular malformations, hemorrhagic processes, or tumors, including, for example, uterine fibromas, primary or secondary liver tumors. For example, vascular occlusion may cause a tumoral necrosis and avoid a more invasive operation. This occlusion technique may also be coupled to delivery of an anticancer agent in the context of chemoembolization. This makes it possible to increase the local concentration of a medicinal product by targeted injection, as well as its residence time in the tumor. In the case of vascular malformations, vascular occlusion makes it possible to normalize blood flow to normal tissues, and aid surgery by limiting the risk of hemorrhage. In hemorrhagic processes, vascular occlusion may lead to a decrease in flow, which promotes healing of the arterial wound. Furthermore, depending on the pathologies treated, embolization may be used for temporary purposes or for permanent purposes.

The commercial embolic agents for vascular occlusion comprise embolization liquids (acrylic adhesives, gels), mechanical devices and particles for embolization. The choice of a specific material depends on many factors, such as the type of lesion to be treated and the type of catheter to be used and the need for temporary or permanent embolization.

The particles for embolization mainly comprise natural and synthetic polymers. Embolic agents of the polymer type offer an advantage as they generally have good biocompatibility with the tissues.

They may be hydrophobic materials. However, the latter are being used less and less in embolization as they are difficult or even impossible to inject in catheters and present a risk of obstruction of the catheter, which makes it necessary for the user to replace the latter, lengthens the procedure and increases the risks thereof.

For example, dry particles of polyvinyl alcohol (PVA) are injected in catheters after being suspended in injectable liquids such as saline solution and iodinated contrast media. They remain rather hydrophobic, even after being suspended, and have a tendency to form aggregates in the syringe, the base and the lumen of the catheter, which occlude the injection catheters. Several technical measures (addition of collagen, albumin, dextran, particles of gelatin sponge, alcohol etc.) have been proposed but without success for preventing these aggregates and obstruction (Derdeyn CP, Moran CJ, Cross DT, Dietrich HH, Dacey RG Jr. Polyvinyl alcohol particle size and suspension characteristics. AJNR Am J Neuroradiol. 1995 Jun-Jul; 16(6):1335-43).

Hydrophilic materials have therefore been considered for embolization, such as microspheres of trisacryl gelatin or gelatin sponges, as they are easier to suspend, are injectable, and cause catheter obstruction less often than hydrophobic materials (C P Derdeyn, V B Graves, M S Salamat and A Rappe, Collagen-coated acrylic microspheres for embolotherapy: in vivo and in vitro characteristics. American Journal of Neuroradiology April 1997, 18 (4) 647-653).

In order to verify the exact location of the embolic particles and to detect reflux in organs that are not targeted, the embolic particles are made radiopaque, i.e. visible in X-ray images. The radiopaque embolic particles may thus be localized in order to detect whether the coverage of a chemotherapy is suitable or not, in order to observe whether the dispersion of the embolic particles in a target zone is homogeneous or heterogeneous, complete or incomplete, or in order to detect whether particles are located outside the target zone.

Embolic particles of radiopaque polymers are described in patent US 4,622,367 and in the work by Horak et al., Biomaterials, 1987, 8, 142. These particles form the basis of acrylate and methacrylate polymers and copolymers and comprise a derivative of aminotriiodobenzoic acid, which is distributed in the matrix network. However, the tri-iodinated molecule is too bulky to be diffused easily in the network and therefore mainly grafts onto the surface of the particle, limiting the transport of water to the interior of the particle, thus leading to loss of the hydrophilic character of the material and consequently loss of the properties of swelling in water. This drawback limits the medical applications of materials of this kind, in particular by making their administration by injection very difficult or even impossible.

The work by Jayakrishnan et al., J. Biomed Mat Res, 1990, 24, 993, describes radiopaque microspheres of the hydrogel type based on PHEMA/iothalamic acid and PHEMA/iopanoic acid copolymers. However, these microspheres are very rigid, and therefore difficultly injectable.

The work by Horak et al., J. Biomed Mat Res, 1997, 34, 183, describes particles of the radiopaque hydrogel type based on PHEMA. The presence of an ionizable group within the structure improves the properties of swelling of the microsphere but these properties remain limited on account of the porous structure of the microsphere. Therefore they are still difficultly injectable.

Patent application US 2009/0297612 describes solid, homogeneous spherical particles of radiopaque copolymers, with controllable swelling properties, and the use thereof in embolization. These particles are based on at least one hydrophilic monomer and at least one radiopaque monomer of general formula (CH_{z}═CR)—CO—R₁. The examples in this application show that the swelling properties decrease when the content of iodinated monomer increases, thus making the microspheres more rigid. Embolization microspheres of this kind are marketed under the name X-Spheres®. It is therefore difficult to obtain nonrigid microspheres comprising a sufficient quantity of iodinated monomer.

Patent application US 2015/0110722 and the work by Duran et al., Theranostics 2016, 6, 28, describe radiopaque particles based on crosslinked PVA and an iodinated compound (triiodobenzyl) . However, these particles have a high density (density 1.21-1.36 g/cm³) and a rigid structure, a reduced water content, leading to difficulty in forming a suspension, very limited injectability or requiring the use of catheters with an inside diameter far greater than the diameter of the microspheres.

In all these examples, it has clearly been observed that the addition of a radiopaque entity or monomer possessing halogenated groups reduces the hydrophilic character of the material considerably. To summarize, the current microspheres loaded with iodine in order to be visible in X-raying are hydrophobic, dense and rigid. As a result, (1) they are difficult to maintain in suspension for the duration of the injection in the catheter, and (2) they often block the catheter, even when their diameter is less than the inside diameter of the catheter (Duran 2016).

There is therefore a great need for preparing microspheres based on a radiopaque polymer which, while comprising halogens (about 5 to 50 mol%), remain hydrophilic and flexible when swollen with water. It is thus desirable for these microspheres to have mechanical properties, in particular a degree of swelling, elasticity and compressibility, suitable for injection via a catheter or a microcatheter and able to regain their original shape after injection while avoiding embolization remote from the target site.

It is also desirable for these microspheres to be able to remain in suspension in mixtures of contrast medium and buffer solution for the duration of injection in the catheter. In fact, to be injectable, the microspheres are generally suspended in a mixture of nonionic iodinated contrast medium and buffer solution. For this purpose, radiologists generally use a solution of contrast medium and optionally saline solution, bicarbonate buffer or phosphate buffer, advantageously a solution of 100% of contrast medium. To ensure injectability, the microspheres must be maintained in suspension homogeneously in this solution. If the microspheres sediment or, conversely, float to the surface of the solution, the resultant suspension is inhomogeneous and unstable, and therefore cannot be injected into the patient.

It is thus advantageous to have microspheres having a suitable density to allow homogeneous suspension in a mixture comprising saline solution, bicarbonate buffer or phosphate buffer with a contrast medium in proportions between 50/50 and 0/100.

Moreover, it is necessary that they can be made visible in magnetic resonance imaging (MRI) and are capable of being loaded with active ingredients.

### SUMMARY OF THE INVENTION

The present invention thus makes it possible to meet these needs and to propose a solution to the various drawbacks encountered in the prior art.

The present invention mainly relates to a polymer comprising a crosslinked matrix, said matrix being based on at least:
a) 20% to 90% of hydrophilic monomer selected from N-vinylpyrrolidone and a monomer of the following formula (I) :

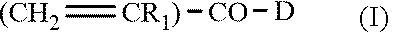

in which:
   D represents O—Z or NH—Z, Z representing (C₁-C6) alkyl, —(CR₂R₃)ₘ—CH₃, —(CH₂—CH₂—0)ₘ—H, —(CH₂—CH₂—0)m—CH₃, —C(R₄OH)ₘ or —(CH₂)ₘ—NR₅R₆ with m representing aninteger from 1 to 30, preferably m is equal to 4 or 5
   R₁, R₂, R₃, R₄, R₅ and R₆ represent, independently of one another, H or a (C₁-C₆) alkyl;
b) 5% to 50% of halogenated radiopaque monomer of the following general formula (II):

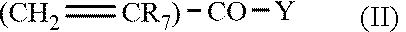

in which
   Y represents O—W, (O—R₈)_{P}—W, (NH—R₈)ₚ—W or NH—W, W representing Ar, L—Ar, and p being an integer between 1 and 10, preferably between 1 and 4 in which:
   Ar represents a (C₅-C₃₆) aryl or (C₅-C₃₆) heteroaryl group, said group being substituted with one, two or three atoms of iodine and/or bromine, and optionally substituted with one to four, preferably two or three, groups selected from (C₁-C₁₀) alkyl, —NRₐR_{b}, —NR_{c}COR_{d}, —COORₑ, —OR_{f}, —OCOR_{g}, —CONR ₕRᵢ, —OCONRⱼRₖ, —NR₁COORₒ, —NRrCONR₅Rₜ, —OCOORᵤ, and —CORᵥ;
   L represents —(CH₂)ₙ—, —(HCCH)ₙ—, -O-, -S-, —SO—,—SO₂—, —OSO₂—, —NR9—, —CO—, —COO—, —OCO—, —OCOO—,—CONR₁₀—, —NR₁₁CO—, —OCONR₁₂—, —NR₁₃COO— or —NR₁₄CONR₁₅—,n being an integer from 1 to 10;
   R₉ to R₁₅ and Rₐ to Rᵥ represent, independently of one another, a hydrogen atom, a (C₁-C₁₀) alkyl, said (C₁-C₁₀)alkyl optionally being substituted with 1 to 10 OH groups, or a group —(CH₂—CH₂—O) q-R′, R′ being a hydrogen atom or a –(C₁-C₆)alkyl and q being an integer between 1 and 10, preferably between 1 and 5;
   R₇ represents H or a (C₁-C₆)alkyl;
   R₈ represents a group selected from (C₁-C₃₆)alkylene, (C₃-C₃₆)cycloalkylene, (C₂-C₃₆)alkenylene, (C₃-C₃₆)cycloalkenylene, (C₂-C₃₆)alkynylene, (C₃-C₃₆)cycloalkynylene, (C₃-C₃₆)arylene and (C₅-C₃₆)heteroarylene,
c) 1% to 15% of nonbiodegradable linear or branched hydrophilic crosslinking agent having groups (CH₂═(CR₁₆) ) – at each of its ends, each R₁₆ independently representing H or a (C₁–C₆)alkyl; and
d) 0.1% to 10% of transfer agent selected from alkyl halides and cycloaliphatic or aliphatic thiols in particular having from 2 to 24 carbon atoms, and optionally having another functional group selected from the amino, hydroxy and carboxy groups, the percentages of the monomers a) to c) being given in moles relative to the total number of moles of monomers, and the percentages of compound d) being given in moles relative to the number of moles of the hydrophilic monomer a).

The inventors discovered that addition of a transfer agent during polymerization of a radiopaque polymer makes it possible to improve the hydrophilicity properties of the microspheres formed by this polymer and thus allows them to be injected. When the transfer agent is not added to the polymer according to the invention, the microspheres obtained are noninjectable, which limits their range of therapeutic application. The polymer according to the invention thus makes it possible to obtain embolization microspheres that are easily injectable and meet all the needs mentioned above.

The present invention further relates to a pharmaceutical composition comprising at least one polymer according to the invention, in association with a pharmaceutically acceptable vehicle, advantageously for administration by injection.

The present invention also relates to a kit comprising a pharmaceutical composition as defined above and at least one means of injection for administration of said composition by the parenteral route.

The present invention also relates to a kit comprising on the one hand a pharmaceutical composition as defined above and on the other hand a contrast agent for imaging by X-ray, by magnetic resonance or by ultrasonography, and optionally at least one means of injection for parenteral administration.

The present invention also relates to a compound with the following general formula (V):

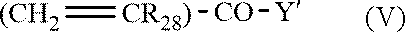

in which
R₂₈ represents H or a (C₁-C₆) alkyl;
Y′ represents, (O—R₂₉)ₜ—W′—Ar′, or NH—W′—Ar′, t being an integer between 1 and 10, preferably between 1 and 4;
R₂₉ represents a group selected from (C₂-C₃₆) alkylene;
W′ represents a single bond, —CONR₃₀—, or —NR₃₁CO—;
Ar′ represents a (C₅-C₃₆) aryl group, said group being substituted with one, two or three atoms of iodine and/or bromine, and optionally substituted with one to four, preferably two or three, groups selected from (C₁-C₁₀)alkyl, —NR₃₂R₃₃, —NR₃₄COR₃₅, —COOR₃₆, —OR₃₇, —OCOR₃₈, —CONR₃₉R₄₀, —OCONR₄₁R₄₂, —NR₄₃COOR₄₄, —NR₄₅CONR₄₆R₄₇, —OCOO R₄₈, and —COR₄₉;
R₃₀ and R₃₁ represent, independently of one another, a hydrogen atom or a (C₁-C₆)alkyl;
R₃₂ to R₄₉ represent, independently of one another, a hydrogen atom, a (C₁-C₁₀)alkyl, said (C₁-C₁₀)alkyl optionally being substituted with 1 to 10 OH groups, or a group —(CH₂—CH₂—O)_{t′}—R″, R″ being a hydrogen atom or a -(C₁-C₆)alkyl and t′ being an integer between 1 and 10, preferably between 1 and 5.

The present invention also relates to the use of the compound of general formula (V) as defined above as a radiopaque halogenated monomer.

### Definitions

The expression “matrix based on” is to be understood as a matrix comprising the mixture and/or the product of reaction between the base constituents used for the heterogeneous polymerization of this matrix, preferably only the product of reaction between the different base constituents used for this matrix, certain of which may be intended to react or are liable to react together or with their close chemical environment, at least partly, during the different steps of the method of manufacture of the matrix, in particular during a polymerization step. Thus, the base constituents are the reactants intended to react together during polymerization of the matrix. The base constituents are therefore introduced into a reaction mixture optionally further comprising a solvent or a mixture of solvents and/or other additives such as at least one salt and/or at least one polymerization initiator and/or at least one stabilizer such as PVA. In the context of the present invention, the reaction mixture comprises at least the monomers a), b), c) and the transfer agent d) mentioned in the present description as base constituents, optionally a polymerization initiator for example such as t-butyl peroxide, benzoyl peroxide, azobiscyanovaleric acid (also called 4,4′-azobis(4-cyanopentanoic) acid), AIBN (azobisisobutyronitrile), or 1,1′-azobis(cyclohexane carbonitrile) or one or more thermal initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenylacetophenone (Irgacure®, 24650-42-8) or 2-methyl-4′-(methylthio)-2-morpholinopropiophenone (Irgacure®, 71868-10-5), and at least one solvent, preferably a solvent mixture comprising an aqueous solvent and an organic solvent such as a nonpolar aprotic solvent, for example a water/toluene mixture.

Thus, according to the present invention, the matrix is at least based on monomers a), b), c) and transfer agent d) mentioned in the present description, these compounds therefore being base constituents.

Thus, in the present description, the expressions similar to “the [base constituent X] is in particular added to the reaction mixture in an amount from YY% to YYY%” and to “the crosslinked matrix is in particular based on the [base constituent X] in an amount from YY% to YYY%” are interpreted similarly. Moreover, expressions similar to “the reaction mixture comprises at least [the base constituent X]” and to “the crosslinked matrix is based on at least [the base constituent X]” are interpreted similarly.

“Organic phase” of the reaction mixture means, in the sense of the present invention, the phase comprising the organic solvent and the compounds soluble in said organic solvent, in particular the monomers, the transfer agent and the polymerization initiator.

“(C_{X}–C_{Y}) alkyl” group means, in the sense of the present invention, a saturated, linear or branched monovalent hydrocarbon-containing chain comprising X to Y carbon atoms, X and Y being integers between 1 and 36, preferably 1 and 18, in particular 1 and 6. As examples, mention may be made of the methyl, ethyl, propyl, isopropyl, butyl, isobutyl, sec-butyl, tert-butyl, pentyl or hexyl groups.

“(C_{X}–C_{Y}) aryl” means, in the sense of the present invention, an aromatic hydrocarbon-containing group, preferably comprising from X to Y carbon atoms, and comprising a ring or several fused rings, X and Y being integers between 5 and 36, preferably 5 and 18, in particular 5 and 10. As examples, mention may be made of the phenyl or naphthyl groups.

“(C_{X}–C_{Y})heteroaryl” means, in the sense of the present invention, an aromatic group comprising X to Y cyclic atoms including one or more heteroatoms, advantageously 1 to 4 and even more advantageously 1 or 2, such as for example sulfur, nitrogen or oxygen atoms, the other cyclic atoms being carbon atoms. X and Y are integers between 5 and 36, preferably 5 and 18, in particular 5 and 10. Examples of heteroaryl groups are the furyl, thienyl, pyrrolyl, pyridinyl, pyrimidinyl, pyrazolyl, imidazolyl, triazolyl, tetrazolyl or indyl groups.

“(C_{X}–C_{Y}) alkylene group” means, in the sense of the present invention, a linear or branched, divalent hydrocarbon-containing chain, comprising X to Y carbon atoms, X and Y being integers between 1 and 36, preferably 1 and 18, in particular 1 and 6. As examples, mention may be made of the methylene, ethylene, propylene, butylene, pentylene or hexylene groups.

“(C_{X}–C_{Y})cycloalkylene group” means, in the sense of the present invention, a saturated, cyclic, divalent hydrocarbon-containing group, comprising from X to Y cyclic carbon atoms, X and Y being integers between 3 and 36, preferably 3 and 18, in particular 3 and 6. As examples, mention may be made of the cyclopropylene, cyclohexylene or cyclopentylene groups.

“(C_{X}–C_{Y})alkenylene group” means, in the sense of the present invention, a linear or branched, divalent hydrocarbon-containing chain, comprising X to Y carbon atoms and at least one double bond, X and Y being integers between 2 and 36, preferably 2 and 18, in particular 2 and 6. As examples, mention may be made of the vinylene (ethenylene) or propenylene groups.

“(C_{X}–C_{Y})cycloalkenylene group” means, in the sense of the present invention, a saturated, cyclic, divalent hydrocarbon-containing group, comprising from X to Y cyclic carbon atoms and at least one double bond, X and Y being integers between 3 and 36, preferably 3 and 18, in particular 3 and 6.

“(C_{X}–C_{Y})alkynylene group” means, in the sense of the present invention, a linear or branched, divalent hydrocarbon-containing chain, comprising X to Y carbon atoms and at least one triple bond, X and Y being integers between 2 and 36, preferably 2 and 18, in particular 2 and 6.

“(C_{X}–C_{Y})cycloalkynylene group” means, in the sense of the present invention, a saturated, cyclic, divalent hydrocarbon-containing group, comprising from X to Y cyclic carbon atoms and at least one triple bond, X and Y being integers between 3 and 36, preferably 3 and 18, in particular 3 and 6.

“(C_{X}–C_{Y})arylene” means, in the sense of the present invention, a divalent aromatic hydrocarbon-containing group, comprising from X to Y carbon atoms, and comprising one or more fused rings, X and Y being integers between 5 and 36, preferably 5 and 18, in particular 5 and 10. As examples, mention may be made of the phenylene group.

“(C_{X}–C_{Y})heteroarylene” means, in the sense of the present invention, a divalent aromatic group, comprising from X to Y cyclic atoms including one or more heteroatoms, advantageously 1 to 4 and even more advantageously 1 or 2, such as for example sulfur, nitrogen or oxygen atoms, the other cyclic atoms being carbon atoms. X and Y are integers between 5 and 36, preferably 5 and 18, in particular 5 and 10.

“Divalent radical” means, in the sense of the present invention, a radical having a valence of 2, i.e. having two covalent, polar covalent or ionic chemical bonds. Said radical may comprise for example carbon atoms and/or oxygen atoms.

“Dry extract” means, in the sense of the present invention, the mass of dry microspheres contained in 1 ml of water-swollen microspheres.

### DETAILED DESCRIPTION

The present invention mainly relates to a polymer comprising a crosslinked matrix, said matrix being based on at least:
a) 20% to 90% of hydrophilic monomer selected from N-vinylpyrrolidone, and a monomer of the following formula (I) :

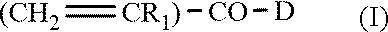

in which:
   D represents O—Z or NH—Z, Z representing (C₁-C6)alkyl, —(CR₂R₃)ₘ—CH₃, —(CH₂—CH₂—0)ₘ—H, —(CH₂—CH₂—0)ₘ—CH₃, —C(R₄OH)ₘ or —(CH₂)ₘ—NR₅R₆ with m representing an integer from 1 to 30;
   R₁, R₂, R₃, R₄, R₅ and R₆ represent, independently of one another, H or a (C₁-C₆)alkyl;
b) 5% to 50% of halogenated radiopaque monomer of the following general formula (II):

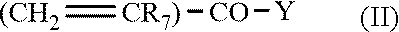

in which
   Y represents O—W, (O—R₈)_{P}—W, (NH—R₈)ₚ—W or NH—W, W representing Ar, L—Ar, and p being an integer between 1 and 10, preferably between 1 and 4 in which:
   Ar represents a (C₅-C₃₆)aryl or (C₅-C₃₆)heteroaryl group, said group being substituted with one, two or three atoms of iodine and/or bromine, and optionally substituted with one to four, preferably two or three, groups selected from (C₁-C₁₀)alkyl, —NRₐR_{b}, —NR_{c}COR_{d}, —COORₑ, —OR_{f}, —OCOR_{g}, —CONR ₕRᵢ, —OCONRⱼRₖ, —NRᵢCOORₒ—, —NRᵣCONR₅Rₜ, —OCOORᵤ, and —CORᵥ;
   L represents —(CH₂)ₙ—, —(HCCH)ₙ—, -O-, -S-, —SO—,—SO₂—, —OSO₂—, —NR₉—, —CO—, —COO—, —OCO—, —OCOO—,—CONR₁₀—, —NR₁₁CO—, —OCONR₁₂—, —NR₁₃COO— or —NR₁₄CONR₁₅—,n being an integer from 1 to 10;
   R₉ to R₁₅ and Rₐ to Rᵥ represent, independently of one another, a hydrogen atom; a (C₁-C₁₀)alkyl, said (C₁-C₁₀)alkyl optionally being substituted with 1 to 10 OH groups; or a group —(CH₂—CH₂—O) q-R′, R′ being a hydrogen atom or a -(C₁-C₆)alkyl and q being an integer between 1 and 10, preferably between 1 and 5;
   R₇ represents H or a (C₁-C₆)alkyl;
   R₈ represents a group selected from (C₁-C₃₆)alkylene, (C₃-C₃₆)cycloalkylene, (C₂-C₃₆)alkenylene, (C₃-C₃₆)cycloalkenylene, (C₂-C₃₆)alkynylene, (C₃-C₃₆)cycloalkynylene, (C₃-C₃₆)arylene and (C₅-C₃₆)heteroarylene.
c) 1% to 15% of nonbiodegradable linear or branched hydrophilic crosslinking agent having groups (CH₂═(CR₁₆) ) – at each of its ends, each R₁₆ independently representing H or a (C₁-C₆)alkyl; and
d) 0.1% to 10% of transfer agent selected from alkyl halides and cycloaliphatic or aliphatic thiols in particular having from 2 to 24 carbon atoms, and optionally having another functional group selected from the amino, hydroxy and carboxy groups, the percentages of the monomers a) to c) being given in moles relative to the total number of moles of monomers and the percentages of compound d) being given in moles relative to the number of moles of the hydrophilic monomer a).

Preferably, the polymer according to the invention is in the form of a spherical particle. The spherical particle is preferably a microsphere.

“Microspheres” means, in the sense of the present invention, spherical particles having a diameter after swelling in the range from 20 to 1200 µm, for example from 20 to 100 µm, from 40 to 150 µm, from 100 to 300 µm, from 300 to 500 µm, from 500 to 700 µm, from 700 to 900 µm or from 900 to 1200 µm, as determined by optical microscopy. The microspheres advantageously have a small enough diameter to be injected by means of needles, a catheter or a microcatheter with an inside diameter in the range from some hundreds of micrometers to more than one millimeter.

The expression “after swelling” signifies that the size of the microspheres is considered after the steps of polymerization and sterilization that take place during their preparation. The sterilization step involves for example passage of the microspheres, after the polymerization step, in an autoclave at high temperature, typically at a temperature above 100° C., preferably at a temperature between 110° C. and 150° C., preferably 121° C. During this sterilization step, the microspheres continue to swell in a controlled manner, i.e. with a controlled degree of swelling. The degree of swelling is defined as:

$$d e g r e e o f s w e l l i n g b y w e i g h t \left(Q\right) = \frac{{m}_{w} \left(g\right) - {m}_{d} \left(g\right)}{m {}_{d} \left(g\right)}$$

where m_{w} is the weight in grams of 1 mL of sedimented microspheres and m_{d} is the weight in grams of 1 ml of sedimented microspheres which have then been lyophilized. “Controlled degree of swelling” means, in the sense of the present invention, that the degree of swelling is reproducible as a function of the batches, in particular that it differs by less than 15% from one batch to another.

“Sedimented microspheres” means, in the sense of the present invention, microspheres that are put into solution in a vessel and are then left for a sufficiently long time without stirring so that they sink to the bottom of the vessel in which they are contained, it thus being possible to remove the supernatant.

“Lyophilized microsphere” means, in the sense of the present invention, microspheres that have undergone freezing followed by dehydration by sublimation.

“Hydrophilic monomer” means, in the sense of the present invention, a monomer having a strong affinity for water, i.e. tending to dissolve in water, to mix with water, to be wetted by water, or capable of swelling in water after polymerization.

The hydrophilic monomer a) of the present invention is selected from N-vinylpyrrolidone, and a monomer of the following formula (I):

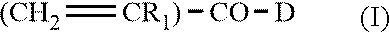

in which:

D represents O—Z or NH—Z, Z representing (C₁-C₆)alkyl, —(CR₂R₃)ₘ—CH₃, —(CH₂—CH₂—O)ₘ—H, —(CH₂—CH₂—O)ₘ—CH₃, —C(R₄OH)ₘ or —(CH₂)ₘ—NR₅R₆ with m preferably representing an integer between 1 and 10, more preferably m is equal to 4 or 5.

Advantageously, the hydrophilic monomer a) according to the invention is selected from the group consisting of N-vinylpyrrolidone, vinyl alcohol, 2-hydroxyethylmethacrylate, sec-butyl acrylate, n-butyl acrylate, t-butyl acrylate, t-butyl methacrylate, methylmethacrylate, N-dimethylaminoethyl(methyl)acrylate, N,N-dimethylaminopropyl- (meth)acrylate, t-butylaminoethyl(methyl)acrylate, N,N-diethylaminoacrylate, poly(ethylene oxide) (meth)acrylate, methoxy poly(ethylene oxide) (meth)acrylate, butoxy poly(ethylene oxide) (meth)acrylate, poly(ethylene glycol) (meth)acrylate, methoxy poly(ethylene glycol) (meth)acrylate, butoxy poly(ethylene glycol) (meth)acrylate, poly(ethylene glycol) methyl ether methacrylate (m-PEGMA), and mixtures thereof.

More advantageously, the hydrophilic monomer a) is poly(ethylene glycol) methyl ether methacrylate (m-PEGMA) .

In the context of the present invention, the hydrophilic monomer a) is in particular added to the reaction mixture in an amount from 20% to 90%, preferably 30% to 80%, preferably from 40% to 70%, in particular from 45% to 65% (mol%), relative to the total number of moles of monomers. Thus, in the context of the present invention, the crosslinked matrix is in particular based on the hydrophilic monomer a) in an amount from 20% to 90%, preferably 30% to 80%, preferably from 40% to 70%, in particular from 45% to 65% (mol%), relative to the total number of moles of monomers.

Radio-opacity refers to the relative inability of electromagnetism, in particular X-rays, to pass through dense materials, which are described as “radiopaque”, appearing opaque/white in a radiographic image. Bearing in mind the complexity of the content in a radiographic or fluoroscopic image, clinicians are sensitive to the quality of the image in regard to the luminosity or the power of the signal from the material in the image. The two main factors contributing to the level of radio-opacity are density and atomic number. Medical devices based on polymers requiring radio-opacity typically use a mixture of polymers that incorporates a small amount, in percentage by weight, of a radiopaque element for example such as a heavy atom such as a halogen, in particular iodine. The capacity of a device to be visualized by fluoroscopy depends on the amount or density of the radiopaque element mixed in with the material. The amount of the radiopaque element in the mixture is generally limited to a small amount as it may have an unfavorable effect on the properties of the material of the base polymer.

In the context of the present invention, the radiopaque monomer is advantageously a monomer of general formula (II) as defined above, in which Y represents NH-W, O—W or (O—R₈)_{P}—W, advantageously NH—W or (O—R₈)ₚ—W, more advantageously (O—R₈)_{P}—W, W representing Ar or L—Ar, p, R₈, L and Ar being as defined above. Preferably, R₈ is a (C₁-C₃₆)alkylene, in particular a (C₁-C₁₈)alkylene, more particularly a (C₁-C₆)alkylene; L represents —OCO—; and Ar represents a (C₅-C₃₆)aryl, in particular a (C₅-C₁₀)aryl, more particularly a phenyl, substituted with one, two or three atoms of iodine and/or bromine, preferably of iodine, and optionally two or three groups selected from —NRₐR_{b}, —NR_{c}COR_{d}, —COORₑ, —OCOR_{g}, —CONRₕRᵢ, —OCONRⱼRₖ, —NR₁COORₒ— and —NRᵣCONR₅Rₜ, preferably —NRₐR_{b}, —NR_{c}COR_{d}. Advantageously, the radiopaque monomer is a monomer of general formula (II) as defined above, in which Y represents NH—W or (O—R₈)ₚ—W, more advantageously (O—R₈)ₚ—W, W representing Ar or L—Ar, and p, R₈, L and Ar being as defined above. Preferably, R₈ is a (C₂-C₃₆)alkylene, in particular a (C₂-C₁₈)alkylene, more particularly a (C₂-C₆)alkylene; L represents —OCO—, —C(O)NR₁₀—, or —NR₁₁C(O)—; and Ar represents a (C₅-C₃₆)aryl, in particular a (C₅-C₁₀)aryl, more particularly a phenyl, substituted with one, two or three atoms of iodine and/or bromine, preferably of iodine, and optionally two or three groups selected from —NRₐR_{b}, —NR_{c}COR_{d}, —COORₑ, —OCOR_{g}, —CONRₕRᵢ, —OCONRⱼRₖ, —NR₁COORₒ— and —NRᵣCONR₅Rₜ, preferably —NRₐR_{b}, —NR_{c}COR_{d} and —C(O)NRₕRᵢ.

Advantageously, Ar represents a (C₅-C₁₀)aryl, more particularly a phenyl, substituted with three atoms of iodine and/or of bromine, preferably of iodine, and optionally two groups selected from (C₁-C₁₀)alkyl, —NRₐR_{b}, —NR_{c}COR_{d}, —COORₑ, —OCOR_{g}, —CONRₕRᵢ, —OCO NRⱼRₖ, —NR₁COORₒ— and —NRᵣCONR₅Rₜ.

Advantageously, Ar represents a phenyl substituted with three atoms of iodine and/or of bromine, preferably of iodine, and optionally two groups selected from (C₁-C₁₀)alkyl, —NRₐR_{b}, —NR_{c}COR_{d}, —COORₑ, —OCOR_{g}, —CONRₕRᵢ, —OCO NRⱼRₖ, —NR₁COORₒ— and —NRᵣCONR₅Rₜ, advantageously from (C₁-C₁₀)alkyl, —NRₐR_{b}, —NR_{c}COR_{d}, —COORₑ, —CONRₕRᵢ, —NR₁COORₒ—and —NRᵣCONR₅Rₜ.

Advantageously, the radiopaque monomer is a monomer of general formula (II) as defined above, in which Y represents O—C₆H₄I, O—C₆H₂I₂, O—C₆H₂I₃, NH—C₆H₄I, NH—C₆H₃I₂, NH—C₆H₂I₃, O—CH₂—CH₂—C(O)—C₆H₄I, O—CH₂—CH₂—O—C(O)—C₆H₃I₂, O—CH₂—CH₂—O—C(O)—C₆H₂I₃, NH—CH₂—CH₂—C(O)—C₆H₄I, NH—CH₂—CH₂—O—C(O)—C₆H₃I₂, or NH—CH₂—CH₂—O—C(O)—C₆H₂I₃, in particular O—C₆H₂I₃, NH—C₆H₂I₃, O—CH₂—CH₂—O—C(O)—C₆H₂I₃, or NH—CH₂—CH₂—O—C(O)—C₆H₂I₃.

Advantageously, the halogenated monomer is selected from the compounds of the following general formula (V):

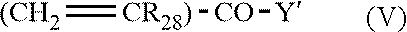

in which
R₂₈ represents H or a (C₁-C₆)alkyl;
Y′ represents (O—R₂₉)ₜ—W′—Ar′, or NH—W′—Ar′, t being an integer between 1 and 10, preferably between 1 and 4;
R₂₉ represents a group selected from (C₂-C₃₆)alkylene;
W′ represents a single bond, —CONR₃₀—, or —NR₃₁CO—;
Ar′ represents a (C₅-C₃₆)aryl group, said group being substituted with one, two or three atoms of iodine and/or bromine, and optionally substituted with one to four, preferably two or three, groups selected from (C₁-C₁₀)alkyl, —NR₃₂R₃₃, —NR₃₄COR₃₅, —COOR₃₆, —OR₃₇, —OCOR₃₈, —CONR₃₉R₄₀, —OCONR₄₁R₄₂, —NR₄₃COOR₄₄, —NR₄₅CONR₄₆R₄₇, —OCOO R₄₈, and —COR₄₉;
R₃₀ and R₃₁ represent, independently of one another, a hydrogen atom or a (C₁-C₆)alkyl;
R₃₂ to R₄₉ represent, independently of one another, a hydrogen atom, a (C₁-C₁₀)alkyl, said (C₁-C₁₀)alkyl optionally being substituted with 1 to 10 OH groups, or a group —(CH₂—CH₂—O)_{t′}—R″, R″ being a hydrogen atom or a -(C₁-C₆)alkyl and t′ being an integer between 1 and 10, preferably between 1 and 5.

Advantageously, R₂₈ represents a (C₁-C₆)alkyl, more advantageously a (C₁-C₃)alkyl, more advantageously a methyl.

Advantageously, R₂₉ represents a (C₂-C₁₈)alkylene, more particularly a (C₂-C₆)alkylene, more advantageously an ethylene.

Advantageously, R₃₀ and R₃₁ represent, independently of one another, a hydrogen atom. Thus, W′ advantageously represents a single bond, —C(O)NH—, or —NHC(O)—.

Advantageously, Ar′ represents a (C₅-C₁₀)aryl, more particularly a phenyl, substituted with one, two or three atoms of iodine and/or bromine, preferably of iodine, and optionally two or three groups selected from (C₁-C₁₀)alkyl, —NR₃₂R₃₃, —NR₃₄C(O)R₃₅, —C(O)OR₃₆, —OR₃₇, —OC(O)R₃₈, —C(O)NR₃₉R₄₀, —OC(O)NR₄₁R₄₂, —NR₄₃C(O)OR₄₄, —NR₄₅C(O)NR₄₆R₄₇, —OC(O)OR₄₈, and —C(O)R₄₉.

Advantageously, Ar′ represents a (C₅-C₁₀)aryl, more particularly a phenyl, substituted with three atoms of iodine and/or of bromine, preferably of iodine, and optionally two groups selected from (C₁-C₁₀)alkyl, —NR₃₂R₃₃, —NR₃₄C (O)R₃₅, —C(O)OR₃₆, —OR₃₇, —OC(O)R₃₈, —C(O)NR₃₉R₄₀, —OC(O)NR₄₁R₄₂, —NR₄₃C(O)OR₄₄, —NR₄₅C(O)NR₄₆R₄₇, —OC(O)OR₄₈, and —C(O)R₄₉.

Advantageously, Ar′ represents a phenyl substituted with three atoms of iodine and/or of bromine, preferably of iodine, and optionally two groups selected from (C₁-C₁₀)alkyl, —NR₃₂R₃₃, —NR₃₄C(O)R₃₅, —C(O)OR₃₆, —OR₃₇, —OC(O)R₃₈, —C(O)NR₃₉R₄₀, —OC(O)NR₄₁R₄₂, —NR₄₃C(O)OR₄₄, —NR₄₅C(O)NR₄₆R₄₇, —OC(O)OR₄₈, and —C(O)R₄₉, advantageously from (C₁-C₁₀)alkyl, —NR₃₂R₃₃, —NR₃₄C(O)R₃₅, —C(O)OR₃₆, —OR₃₇, —C(O)NR₃₉R₄₀, —NR₄₃C(O)OR₄₄, NR₄₅C(O)NR₄₆R₄₇, —OC(O)OR₄₈, and —C(O)R₄₉.

Advantageously, the halogenated monomer is selected from the following compounds:

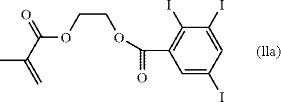 [SMILES: C=C(C)C(=O)OCCOC(=O)c1cc(I)cc(I)c1I]

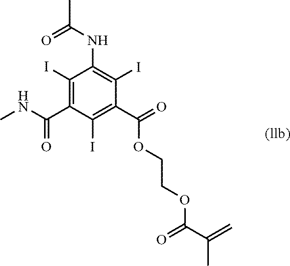 [SMILES: C=C(C)C(=O)OCCOC(=O)c1c(I)c(NC(C)=O)c(I)c(C(=O)NC)c1I]

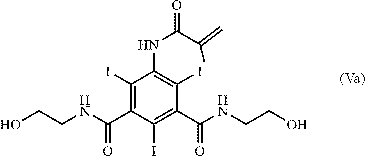 [SMILES: C=C(C)C(=O)Nc1c(I)c(C(=O)NCCO)c(I)c(C(=O)NCCO)c1I]

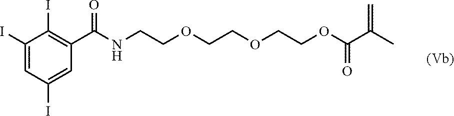 [SMILES: C=C(C)C(=O)OCCOCCOCCNC(=O)c1cc(I)cc(I)c1I]

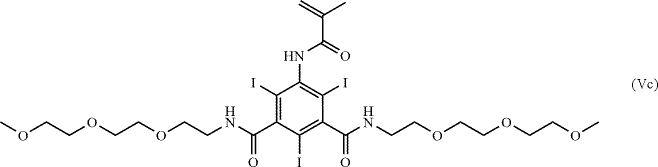

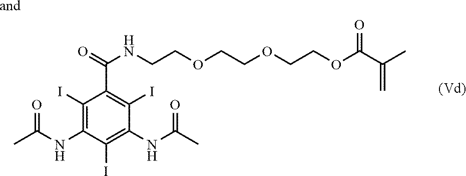

Advantageously, the halogenated monomer is selected from the following compounds:

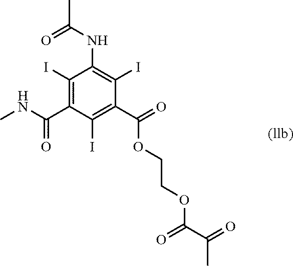 [SMILES: CNC(=O)c1c(I)c(NC(C)=O)c(I)c(C(=O)OCCOC(=O)C(C)=O)c1I]

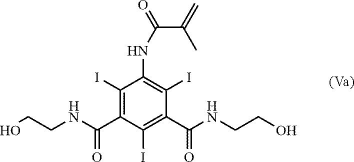 [SMILES: C=C(C)C(=O)Nc1c(I)c(C(=O)NCCO)c(I)c(C(=O)NCCO)c1I]

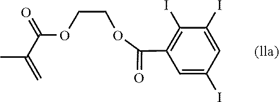 [SMILES: C=C(C)C(=O)OCCOC(=O)c1cc(I)cc(I)c1I]

and

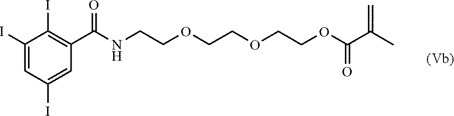 [SMILES: C=C(C)C(=O)OCCOCCOCCNC(=O)c1cc(I)cc(I)c1I]

More advantageously, the radiopaque monomer is the (tri-iodobenzoyl)oxo ethyl methacrylate (MAOETIB) of the following formula (IIa):

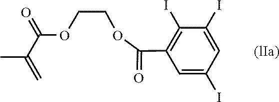 [SMILES: C=C(C)C(=O)OCCOC(=O)c1cc(I)cc(I)c1I]

or 2-(2-(2-(2,3,5-triiodobenzamido)ethoxy)ethyl methacrylate of the following formula:

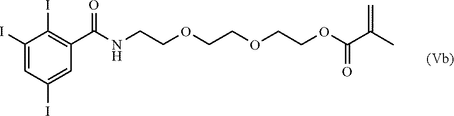 [SMILES: C=C(C)C(=O)OCCOCCOCCNC(=O)c1cc(I)cc(I)c1I]

In the context of the present invention, the radiopaque monomer is in particular added to the reaction mixture in an amount from 5% to 50%, in particular in an amount greater than 7% and less than or equal to 50%, in particular in an amount greater than 10% and less than or equal to 50%, more particularly in an amount greater than 15% and less than or equal to 50%, preferably in an amount greater than 15% and less than or equal to 35%, and in particular from 20% to 30% (mol%), relative to the total number of moles of monomers.

“Crosslinking monomer” means, in the sense of the present invention, a monomer at least bifunctional but also multifunctional possessing a double bond at each polymerizable end. The crosslinking monomer, in combination with the other monomers in the mixture, allows formation of a crosslinked network. The structure and the amount of crosslinking monomer(s) in the mixture of monomers can easily be selected by a person skilled in the art to provide the desired crosslink density. The crosslinking agent is also advantageous for the stability of the microspheres. The crosslinking agent prevents the microspheres being able to dissolve in any solvent. The crosslinking agent also makes it possible to improve the compressibility of the microspheres, which is favorable to embolization.

“Nonbiodegradable hydrophilic crosslinking agent” means, in the sense of the present invention, a crosslinking agent as defined above, having a strong affinity for water and that cannot be degraded in the physiological conditions of the body of a mammal, in particular the human body. In fact, biodegradation of a molecule is permitted when the latter contains sufficient functional sites that can be cleaved in physiological conditions, in particular by the endogenous enzymes in the body of a mammal, in particular in the human body, and/or at physiological pH (generally around 7.4). The functional sites that are cleavable in physiological conditions are in particular amide bonds, ester bonds and acetals. A molecule comprising an insufficient number of said functional sites will therefore be regarded as nonbiodegradable. In the context of the present invention, the crosslinking monomer contains fewer than 20 functional sites that are cleavable in physiological conditions, preferably fewer than 15 sites, more preferably fewer than 10 sites, even more preferably fewer than 5 sites.

The nonbiodegradable linear or branched hydrophilic crosslinking agent is in particular a nonbiodegradable crosslinking agent that is soluble in an organic solvent and comprises diacrylate, methacrylate, acrylamide, and/or methacrylamide polymerizable groups.

Advantageously, the crosslinking agent has (CH₂═(CR₁₆) ) CO— or (CH₂═(CR₁₆) ) CO—O— groups at its at least two ends, each R₁₆ independently representing H or a (C₁-C₆)alkyl; advantageously the radicals R₁₆ are identical and represent H or (C₁-C₆)alkyl.

In particular, the crosslinking agent is of the following general formula (IIIa) or (IIIb):

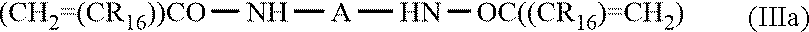

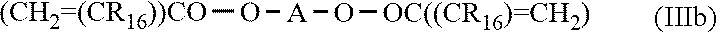

in which each R₁₆ independently represents H or a (C₁-C₆)alkyl, advantageously the radicals R₁₆ are identical and represent H or (C₁-C₆)alkyl; and

A represents, alone or with at least one of the atoms to which it is bound, a (C₁-C₆)alkylene, a polyethylene glycol (PEG), a polysiloxane, a poly(dimethylsiloxane) (PDMS), a polyglycerol ester (PGE) or a bisphenol A.

Advantageously, the crosslinking agent is of the following general formula (IIa) or (IIb):

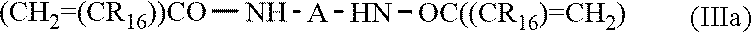

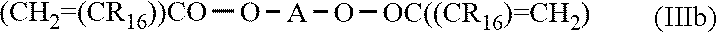

in which, each R₁₆ independently represents H or a (C₁-C₆)alkyl, advantageously the radicals R₁₆ are identical and represent H or (C₁-C₆)alkyl; and

A preferably represents, alone or with at least one of the atoms to which it is bound, a (C₁-C₆)alkylene or a polyethylene glycol (PEG), preferably a polyethylene glycol (PEG).

In the context of the definitions of A given above, the polyethylene glycol has a length in the range from 200 to 10 000 g/mol, preferably from 200 to 2000 g/mol, more preferably from 500 to 1000 g/mol.

As examples of crosslinking monomer usable in the context of the present invention, mention may be made (without being limiting) of: 1,4-butanediol diacrylate, pentaerythritol tetraacrylate, methylenebisacrylamide, glycerol 1,3-diglycerolate diacrylate and poly(ethylene glycol)dimethacrylate (PEGDMA).

Advantageously, the crosslinking monomer is poly(ethylene glycol)dimethacrylate (PEGDMA), the polyethylene glycol unit having a length in the range from 200 to 10 000 g/mol, preferably from 200 to 2000 g/mol, more preferably from 500 to 1000 g/mol.

In the context of the present invention, the crosslinking monomer is in particular added to the reaction mixture in an amount from 1% to 15%, preferably from 2% to 10%, in particular 2% to 7%, more particularly 2% to 5% (mol%), relative to the total number of moles of monomers.

In the context of the present invention, “transfer agent” means a chemical compound possessing at least one weak chemical bond. This agent reacts with the radical site of a growing polymer chain and stops chain growth. In the chain transfer process, the radical is transferred temporarily to the transfer agent, which restarts growth by transferring the radical to another polymer or monomer.

In the context of the present invention, the use of a transfer agent for obtaining the polymer according to the invention makes it possible to preserve its hydrophilicity despite addition of the radiopaque monomer, and thus allows injection of the microspheres. This makes it possible to obtain a more homogeneous polymer network, with improved elastic properties and thus improving the swelling properties.

Advantageously, said chain transfer agent is selected from the group consisting of the monofunctional or polyfunctional thiols, and the alkyl halides.

The alkyl halides that can be used as transfer agent include in particular bromotrichloromethane, tetrachloromethane and tetrabromomethane.

Particularly advantageously, said chain transfer agent is an aliphatic or cycloaliphatic thiol typically having from 2 to about 24 carbon atoms, preferably 2 to 12 carbon atoms, more preferably 6 carbon atoms, and optionally having an additional functional group selected from the amino, hydroxy and carboxy groups.

Examples of particularly preferred chain transfer agents are thioglycolic acid, 2-mercaptoethanol, dodecanethiol, hexanethiol, and mixtures thereof, preferably hexanethiol.

In the context of the present invention, the transfer agent is in particular added to the reaction mixture in an amount from 0.1% to 10%, preferably from 0.5% to 8%, more advantageously from 1.5% to 6% and in particular from 1.5% to 4.5% (mol%), and in particular 3 mol%, relative to the number of moles of hydrophilic monomer a).

In a particular embodiment according to the invention, the crosslinked polymer matrix of the microspheres is solely based on the base constituents a), b), c) and d) as defined above, in the aforementioned proportions of monomers and transfer agent, no other base constituent being added to the reaction mixture. It is thus clear that the sum of the aforementioned proportions of monomers a), b) and c) must be equal to 100%.

According to a particular aspect of the invention, the matrix of the polymer according to the invention is moreover based on at least one ionized or ionizable monomer, of the following formula (IV):

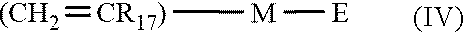

in which:
R₁₇ represents H or a (C₁-C₆)alkyl;
M represents a single bond or a divalent radical having from 1 to 20 carbon atoms, preferably a single bond;
E represents an ionized or ionizable group, E advantageously being selected from the group consisting of —COOH, —COO—, —SO₃H, —SO₃⁻, —PO₄H₂, —PO₄H⁻, —PO₄²⁻, —NR₁₈R₁₉, and —NR₂₀R₂₁R₂₂⁺,
R₁₈, R₁₉, R₂₀, R₂₁ and R₂₂ represent, independently of one another, H or a (C₁-C₆)alkyl.

“Ionized or ionizable group” means, in the sense of the present invention, a group that is charged or may be in charged form (in the form of an ion), i.e. bearing at least one positive or negative charge, depending on the pH of the medium. For example, the COOH group may be ionized in the form COO⁻ and the NH₂ group may be in the ionized form NH₃⁺.

The introduction of an ionized or ionizable monomer into the reaction mixture makes it possible to increase the hydrophilicity of the resultant microspheres, thus increasing the degree of swelling of said microspheres, further facilitating their injection via catheters and microcatheters. Moreover, the presence of an ionized or ionizable monomer allows loading of active substances within the microsphere.

Preferably, the ionized or ionizable monomer is a cationic monomer, advantageously selected from the group consisting of (methacryloyloxy)ethylphosphorylcholine, 2-(dimethylamino)ethyl (meth)acrylate, (2-(diethylamino)ethyl) (meth)acrylate and 2-((meth)acryloyloxy)ethyl)-trimethylammonium chloride; advantageously, the cationic monomer is (diethylamino)ethyl (meth)acrylate. Advantageously, the crosslinked matrix according to the invention is based on an aforementioned cationic monomer in amounts between 1 and 40 mol% relative to the total number of moles of monomers. Preferably, the crosslinked matrix according to the invention is based on an ionized or ionizable monomer in amounts between 5% and 15%, preferably 10 mol% relative to the total number of moles of monomers, when the resultant microspheres are not intended to be loaded with an active substance. According to another embodiment, when the microspheres are intended to be loaded with an active substance, the crosslinked matrix according to the invention is obtained by adding to the reaction mixture between 20% and 40%, preferably by adding to the reaction mixture 20 to 30 mol% of ionized or ionizable monomer relative to the total number of moles of monomers.

In another advantageous embodiment, the ionized or ionizable monomer is an anionic monomer advantageously selected from the group consisting of acrylic acid, methacrylic acid, 2-carboxyethyl acrylate, the 2-oligomers of carboxyethyl acrylate, 3-sulfopropyl (meth)acrylate, the potassium salt and the hydroxide of 2-((methacryloyloxy)ethyl)dimethyl-(3-sulfopropyl)ammonium. Advantageously, the crosslinked matrix according to the invention is based on an aforementioned anionic monomer in amounts between 1 and 40 mol% based on the total amount of monomers. Preferably, the crosslinked matrix according to the invention is based on ionized or ionizable monomer in amounts between 5% and 15%, preferably 10 mol% based on the total amount of monomers, when the resultant microspheres are not intended to be loaded with an active substance. According to another embodiment, when the microspheres are intended to be loaded with an active substance, the crosslinked matrix according to the invention is based on ionized or ionizable monomer in amounts between 20% and 40%, preferably 20% to 30% of ionized or ionizable monomer based on the total amount of monomers.

Particularly advantageously, the ionized or ionizable monomer is methacrylic acid (MA). Advantageously, the crosslinked matrix according to the invention is based on methacrylic acid (MA) in amounts between 10 and 30 mol% based on the total amount of monomers.

In the context of the present invention, the crosslinked matrix according to the invention is moreover based on at least one colored monomer to improve its visibility to the naked eye. This makes it possible in particular to check prior to injection that the suspension of polymers is properly homogeneous in the syringe and to control the rate of injection.

Thus, according to a particular embodiment, the matrix of the polymer according to the invention is moreover based on at least one colored monomer of the following general formula (VI):

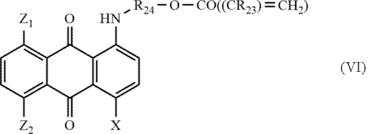

in which,
Z₁ and Z₂ represent, independently of one another, H or OR₂₅, R₂₅ representing H or a (C₁-C₆)alkyl, advantageously Z₁ and Z₂ represent H;
X represents H or a halogen such as Cl, advantageously H;
R₂₃ represents H or a (C₁-C₆)alkyl, advantageously a (C₁-C₆)alkyl, in particular a methyl; and
R₂₄ represents a group selected from linear or branched (C₁-C₆)alkylene, (C₅-C₃₆)arylene, (C₅-C₃₆)arylene-O-R₂₆, (C₅-C₃₆)heteroarylene and (C₅-C₃₆)heteroarylene-O-R₂₇, R₂₆ and R₂₇ representing a (C₁-C₆)alkyl or a (C₁-C₆)alkylene, advantageously R₂₄ represents a —C₆H₄—O—(CH₂)₂— or —C(CH₃)₂—CH₂— group.

Advantageously, the colored monomer is of the following formula (VIa) or (VIb):

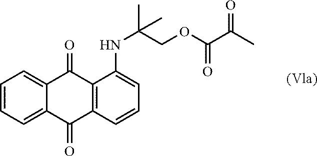 [SMILES: CC(=O)C(=O)OCC(C)(C)Nc1cccc2c1C(=O)c1ccccc1C2=O]

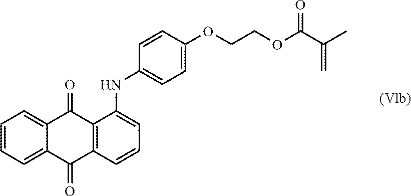 [SMILES: C=C(C)C(=O)OCCOc1ccc(Nc2cccc3c2C(=O)c2ccccc2C3=O)cc1]

More advantageously, the colored monomer is of the above formula (VIb).

In the context of the present invention, the colored monomer is in particular added to the reaction mixture in an amount from 0% to 1%, preferably from 0% to 0.5%, more particularly from 0.02% to 0.2%, and even more particularly from 0.04% to 0.1% (mol%), relative to the total number of moles of monomers.

Magnetic resonance imaging (MRI) is used in the medical setting for supplying images in two-dimensional section of the internal structures of a patient’s body without exposing them to harmful radiation. The matrix of the polymer according to the invention may in particular be based on particles allowing the polymer to be made visible using magnetic resonance imaging (MRI).

Thus, advantageously, the matrix of the polymer according to the invention is moreover based on at least one agent that is visible in magnetic resonance imaging (MRI) such as nanoparticles of iron oxide, gadolinium chelates or magnesium chelates, advantageously nanoparticles of iron oxide such as USPIOs (Ultra Small Super Paramagnetic Iron Oxide or Ultra Small Paramagnetic Iron Oxides, i.e magnetic particles based on an iron compound having superparamagnetic characteristics that make them visible in MRI).

In the context of the present invention, the particles that are visible in MRI are advantageously added to the reaction mixture in an amount from 0% to 10%, preferably from 0.5% to 8%, more preferably from 0.5% to 5%, in particular 1%, by volume of organic phase.

In the context of the present invention, when the matrix of the polymer does not comprise ionized or ionizable monomer as base constituent, it is advantageously based on:
34.5% to 84%, preferably 64.9% to 77.98% of hydrophilic monomer a);
More than 15% to 50%, preferably 20% to 30% of radiopaque monomer b);
1% to 15%, preferably 2% to 5% of nonbiodegradable hydrophilic crosslinking agent c);
1.5% to 4.5% of transfer agent d), preferably 3%;
0% to 0.5% of colored monomer, preferably 0.02% to 0.1%; and
0% to 10% of particles visible in MRI, preferably 1% to 5%,
each of the monomers mentioned and the nature of their associated percentages being as defined above in the present description. It is clear that the sum of the aforementioned percentages of monomers must be equal to 100%.

In the context of the present invention, when the matrix of the polymer does not comprise ionized or ionizable monomer as base constituent, it is advantageously based on:
74.5% to 78% of hydrophilic monomer a)
20% of radiopaque monomer b);
2% to 5% of nonbiodegradable hydrophilic crosslinking agent c);
1.5% to 4.5% of transfer agent d);
0% to 0.5% of colored monomer; and
0% to 10% of particles visible in MRI,
each of the monomers mentioned and the nature of their associated percentages being as defined above in the present description. It is clear that the sum of the aforementioned percentages of monomers must be equal to 100%.

In the context of the present invention, when the matrix of the polymer comprises at least one ionized or ionizable monomer as base constituent, it is advantageously based on:
34.9% to 67.98%, preferably 34.96% to 67.96%, of hydrophilic monomer a)
20% to 30% of radiopaque monomer b);
2% to 5% of nonbiodegradable hydrophilic crosslinking agent c);
1.5% to 3% of transfer agent d);
10% to 30% of ionizable or charged monomer;
0.02% to 0.1%, preferably 0.04%, of colored monomer; and
0% to 10% of particles visible in MRI,
each of the monomers mentioned and the nature of their associated percentages being as defined above in the present description. It is clear that the sum of theaforementioned percentages of monomers must be equal to 100%.

The polymer according to the invention can easily be synthesized by many methods that are familiar to a person skilled in the art. As an example, the polymer according to the invention can be obtained by suspension polymerization as described below and in the examples.

Direct suspension can take place as follows:
(a) mix or stir a reaction mixture comprising:
   (i) at least one hydrophilic monomer a) as defined above, at least one radiopaque monomer b) as defined above, at least one nonbiodegradable hydrophilic crosslinking agent c) as defined above, and at least one transfer agent d) as defined above;
   (ii) a polymerization initiator present in amounts from 0.1 to about 2 parts by weight to 100 parts by weight of the monomers;
   (iii) a surfactant in an amount not greater than about 5 parts by weight to 100 parts by weight of aqueous phase, preferably not greater than about 3 parts by weight and most preferably in the range from 0.2 to 1.5 parts by weight; and
   (iv) water to form an oil-in-water suspension; and
(b) polymerize the base constituents.

In this direct suspension method, the surfactant may be selected from the group consisting of hydroxyethyl cellulose, polyvinyl alcohol (PVA), polyvinylpyrrolidone, polyethylene oxide, polyethylene glycol and Polysorbate 20 (Tween® 20); preferably it is PVA.

The microspheres thus obtained are then washed and calibrated by techniques that are familiar to a person skilled in the art.

An inverse suspension may be prepared as follows:
(a) mix or stir a reaction mixture comprising:
   (i) at least one hydrophilic monomer a) as defined above, at least one radiopaque monomer b) as defined above, at least one nonbiodegradable hydrophilic crosslinking agent c) as defined above, and at least one transfer agent d) as defined above;
   (ii) a polymerization initiator present in amounts from 0.1 to about 2 parts by weight to 100 parts by weight of the monomers;
   (iii) a surfactant in an amount not greater than about 10 parts by weight to 100 parts by weight of the oily phase, preferably not greater than about 8 parts by weight to 100 parts by weight of the oily phase and most preferably in the range from 3 to 7 parts by weight to 100 parts by weight of the oily phase; and
   (iv) oil to form a water-in-oil suspension; and
(b) polymerize the base constituents.

In the aforementioned methods, the polymerization initiator may in particular be t-butyl peroxide, benzoyl peroxide, azobiscyanovaleric acid (also called 4,4′-azobis(4-cyanopentanoic) acid), AIBN (azobisisobutyronitrile), or 1,1′-azobis(cyclohexane carbonitrile) or one or more thermal initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenylacetophenone (Irgacure®, 24650-42-8) or 2-methyl-4′-(methylthio)-2-morpholinopropiophenone (Irgacure®, 71868-10-5).

In this method of inverse suspension, the surfactant may be selected from the group consisting of sorbitan esters such as sorbitan monolaurate (Span® 20), sorbitan monopalmitate (Span® 40), sorbitan monooleate (Span® 80), and sorbitan trioleate (Span® 85), hydroxyethyl cellulose, mixture of glyceryl stearate and PEG stearate (Arlacel®) and cellulose acetate.

The oil used in the method described above may be selected from paraffin oil, silicone oil and the organic solvents such as hexane, cyclohexane, ethyl acetate or butyl acetate.

When the polymer according to the invention is obtained on the basis of polymerization of at least one ionized or ionizable monomer, a medicinal product, an active substance, a diagnostic agent or macromolecules may also be loaded on the polymer, i.e. adsorbed on the polymer by noncovalent interactions, optionally in the presence of pharmaceutically acceptable excipient(s) familiar to a person skilled in the art. This particular manner of trapping the medicinal products or the active substances is called physical encapsulation. No particular requirement is imposed on the medicinal product or the active substance to be loaded.

Loading may be done by many methods that are familiar to a person skilled in the art such as passive adsorption (swelling of the polymer in a solution of medicinal product) or by ionic interaction. These methods are described for example in international application WO 2012/120138, in particular from page 22, line 20 to page 26, line 7. The efficiency of encapsulation mainly depends on the compatibility between the two structures and/or favorable interactions.

In the context of the present invention, the polymer may be loaded with a medicinal product, an active substance or a diagnostic agent and thus allow their release at a target site, said target site being inside a mammal’s body, in particular inside a human body. Monitoring of the loaded polymer by X-raying or by MRI makes it possible to ensure that release of the medicinal product/active substance/diagnostic agent takes place at the desired specific site. The polymer according to the invention may therefore be loaded with a medicinal product or an active substance or a diagnostic agent, advantageously having a molecular weight below 5000 Da, typically below 1000 Da, the medicinal product or the active substance advantageously being selected from the group consisting of anti-inflammatory agents, local anesthetics, analgesics, antibiotics, anticancer agents, steroids, antiseptics and a mixture thereof.

Preferably, the polymer according to the invention may be loaded with an anticancer agent.

The anticancer agent is preferably selected from anthracyclines such as doxorubicin, epirubicin or idarubicin, platinum complexes, compounds related to the anthracyclines such as mitoxantrone and nemorubicin, antibiotics such as mitomycin C (Ametycine®), bleomycin and actinomycin D, other antineoplastic compounds such as irinotecan, 5-fluoro-uracil (Adrucil®), sorafenib (Nevaxar®), sunitinib (Sutent®), regorafenib, brivanib, orantinib, linsitinib, erlotinib, cabozantinib, foretinib, tivantinib, fotemustine, tauromustine (TCNU), carmustine, cytosine C, cyclophosphonamide, cytosine arabinoside (or cytarabine), paclitaxel, docetaxel, methotrexate, everolimus (Afinitor®), PEG-arginine deiminase, the tegafur/gimeracil/oteracil combination (Teysuno®), muparfostat, peretinoine, gemcitabine, bevacizumab (Avastin®), ramucirumab, floxuridine, immunostimulants such as GM-CSF (granulocyte-macrophage colony-stimulating factor) and its recombinant forms: molgramostim or sargramostim (Leukine®), OK-432 (Picibanil®), interleukin-2, interleukin-4 and tumor necrosis factor-alpha (TNFalpha), antibodies, radioelements, complexes of these radioelements with chelates, nucleic acid sequences and a mixture of one or more of these compounds (preferably a mixture of one or more anthracyclines).

Preferably, the anticancer agent is selected from anthracyclines, immunostimulants, platinum complexes, antineoplastics and mixtures thereof.

Even more preferably, the anticancer agent is selected from anthracyclines, antibodies, antineoplastics and mixtures thereof.

The antibodies are for example selected from the anti-PD-1, anti-PD-L1, anti-CTLA-4, anti-CEA (CarcinoEmbryonic Antigen) or a mixture thereof.

The anti-PD-1 are for example nivolumab or pembrolizumab.

The anti-PD-L1 are for example avelumab, durvalumab or atezolizumab.

The anti-CTLA-4 are for example ipilimumab or tremelimumab.

Even more advantageously, the anticancer agent is selected from the group consisting of paclitaxel, doxorubicin, epirubicin, idarubicin, irinotecan, GM-CSF (granulocyte-macrophage colony-stimulating factor), tumor necrosis factor-alpha (TNFalpha), antibodies, and mixtures thereof.

Preferably the local anesthetic is selected from lidocaine, bupivacaine and mixtures thereof.

The anti-inflammatory may be selected from ibuprofen, niflumic acid, dexamethasone, naproxen and mixtures thereof.

In the context of the present invention, the polymer may be loaded, in particular by extemporaneous adsorption, with macromolecules selected from the group consisting of enzymes, antibodies, cytokines, growth factors, clotting factors, hormones, plasmids, antisense oligonucleotides, siRNA, ribozymes, DNA enzyme (also called DNAzyme), aptamers, anti-inflammatory proteins, bone morphogenetic proteins (BMP), pro-angiogenic factors, vascular endothelial growth factors (VEGF) and TGF-beta, and angiogenesis inhibitors or antityrosine kinases and mixtures thereof.

The anti-inflammatory proteins are for example infliximab or rilonacept and a mixture thereof.

The pro-angiogenic factors are for example fibroblast growth factors (FGF) and a mixture thereof.

The angiogenesis inhibitors are for example bevacizumab, ramucirumab, nesvacumab, olaratumab, vanucizumab, rilotumumab, emibetuzumab, aflibercept, ficlatuzumab, pegaptanib and mixtures thereof.

The antityrosine kinases are for example lenvatinib, sorafenib, sunitinib, pazopanib, vandetanib, axitinib, regorafenib, cabozantinib, fruquintinib, nintedanib, anlotinib, motesanib, cediranib, sulfatinib, dovetinib, linifanib and mixtures thereof.

Advantageously, the polymer may be loaded with macromolecules selected from antityrosine kinases, TGF-beta, angiogenesis inhibitors and mixtures thereof.

In a second aspect, the invention relates to a pharmaceutical composition comprising at least one polymer according to the invention, in association with a pharmaceutically acceptable vehicle, advantageously for administration by injection.

An example of pharmaceutically acceptable vehicle comprises, but is not limited to, water for injection, saline solution also called physiological serum, starch, hydrogel, polyvinylpyrrolidone, polysaccharide, ester of hyaluronic acid, plasma, a contrast agent for imaging by X-ray, by magnetic resonance or by ultrasonography, a buffering agent, a preservative, a gelling agent, a surfactant, or a mixture thereof. Advantageously, the pharmaceutically acceptable vehicle is saline solution, water for injection, a contrast agent for imaging by X-ray, by magnetic resonance or by ultrasonography or a mixture thereof. More advantageously, the pharmaceutically acceptable vehicle is a contrast agent for imaging by X-ray, by magnetic resonance or by ultrasonography, saline solution, or a mixture of saline solution and a contrast agent for imaging by X-ray, by magnetic resonance or by ultrasonography.

According to the present invention, the contrast agent is preferably a contrast agent for X-ray imaging. It is advantageously one or more nonionic iodinated water-soluble contrast agents, such as for example iobitridol (Xenetix®), iopamidol (Iopamiron®, Isovue®), iomeprol (Iomeron®), ioversol (Optiray®, Optiject®), iohexol (Omnipaque®), iopentol (Imagopaque®), ioxitol (Oxylan®), iopromide (Ultravist®), metrizamide (Amipaque®), iosarcol (Melitrast®), iotrolan (Isovist®), iodixanol (Visipaque®), iosimenol and iosimide (Univist®) and a mixture thereof.

According to another embodiment, the contrast agent is a contrast agent for magnetic resonance imaging (MRI). It is advantageously gadolinium chelates (Dotarem®).

According to another embodiment, the contrast agent is a contrast agent for imaging by ultrasonography. It is advantageously sulfur hexafluoride (Sonovue®).

In a particular embodiment of the present invention, the pharmaceutical composition comprises the polymer according to the invention, in association with saline solution, said composition being intended to be mixed with at least one contrast agent for imaging by X-ray, by magnetic resonance or by ultrasonography as defined above, in particular for X-ray imaging, before administration by injection, said mixing leading to suspension of the microspheres obtained from the polymer according to the invention.

In a particular embodiment according to the invention, the pharmaceutical composition according to the invention comprises the polymer according to the invention, in association with a mixture of saline solution and a contrast agent as defined above, the saline solution and the contrast agent being present in proportions from 50/50 to 0/100, advantageously from 40/60 to 0/100, preferably from 30/70 to 0/100.

In another particular embodiment according to the invention, the pharmaceutical composition according to the invention comprises the polymer according to the invention, in association with only one or more contrast agents as defined above, in particular one or more contrast agents for X-ray imaging as defined above.

The pharmaceutical composition must have an acceptable viscosity for injection.

The fields of application of the radiopaque polymer according to the invention comprise in particular embolization and chemoembolization.

The polymer according to the invention may, as was stated above, be used for various biomedical purposes, which means that it must be compatible with the body of a mammal and in particular with the human body. More particularly, suitable biomedical materials do not have hemolytic properties.

The present invention further relates to the specific use of a transfer agent in the polymerization of a radiopaque polymer to allow injection of said radiopaque polymer, in particular injection in a catheter or a microcatheter with an inside diameter in the range from some hundreds of micrometers to more than one millimeter. The present invention also relates to the specific use of a transfer agent in the polymerization of a radiopaque polymer for improving the hydrophilicity and the swelling properties in water of said polymer and thus promoting its injection. Said transfer agent is in particular as defined above and in the contents as defined above, and is in particular selected from the cycloaliphatic or aliphatic thiols in particular having from 2 to 24 carbon atoms, and optionally having another functional group selected from the amino, hydroxy and carboxy groups.

The present invention also relates to a kit comprising a pharmaceutical composition as defined above and at least one means of injection of said composition, for parenteral administration of said composition. According to the present invention, “means of injection” means any means allowing administration by the parenteral route. Advantageously, said means of injection is one or more syringes, which may be prefilled, and/or one or more catheters or microcatheters.

Advantageously, the pharmaceutical composition contained in said kit comprises the polymer according to the present invention in association with saline solution, one or more contrast agents as defined above, in particular one or more contrast agents for X-ray imaging as defined above, or a mixture thereof. More advantageously, said pharmaceutical composition comprises the polymer according to the present invention in association with a mixture of saline solution and one or more contrast agents as defined above, in particular one or more contrast agents for X-ray imaging as defined above, in proportions between 50/50 and 0/100, advantageously between 40/60 and 0/100, preferably from 30/70 to 0/100.

Advantageously, the one or more means of injection contained in the kit according to the invention is (are) suitable for parenteral administration of the pharmaceutical composition according to the invention. Thus, the size of the syringe (s) or of the (micro)catheter(s) will be adapted as a function of the size of the microspheres obtained from the polymer according to the invention and the volume to be injected for embolization, the size of the microspheres itself being selected as a function of the size of the vessel to be embolized.

A person skilled in the art will know how to select the appropriate size of the microspheres and therefore the appropriate means of injection.

The present invention also relates to a kit comprising on the one hand a pharmaceutical composition as defined above and on the other hand at least one contrast agent for imaging by X-ray, by magnetic resonance or by ultrasonography, and optionally at least one means of injection for parenteral administration. The means of injection is as defined above.

In said kit, the pharmaceutical composition and the contrast agent are packaged separately and are intended to be mixed just before administration by injection.

In said kit, the at least one contrast agent is as defined above in the description. In particular, the at least one contrast agent is a contrast agent for X-ray imaging as defined above in the description.

In said kit, the pharmaceutical composition advantageously comprises the polymer according to the present invention in association with a pharmaceutically acceptable vehicle for administration by injection. Said pharmaceutically acceptable vehicle may be for example, but is not limited to, water for injection, saline solution, starch, hydrogel, polyvinylpyrrolidone, polysaccharide, ester of hyaluronic acid and/or plasma. Preferably, in said kit, the pharmaceutical composition advantageously comprises the polymer according to the present invention in association with saline solution or water for injection.

In said kit, the pharmaceutical composition is advantageously packaged directly in a means of injection, in particular in a syringe, suitable for injection of embolization microspheres by the parenteral route.

In said kit, the contrast agent is advantageously packaged in a vial or directly in a means of injection, in particular a syringe, in particular suitable for injection of embolization microspheres by the parenteral route.

In said kit, the proportions of pharmaceutically acceptable vehicle / contrast agent are between 50/50 and 0/100, advantageously between 40/60 and 0/100, preferably from 30/70 to 0/100.

The present invention also relates to a compound with the following general formula (V):

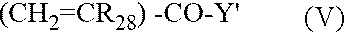

in which
R₂₈ represents H or a (C₁-C₆)alkyl;
Y′ represents (O—R₂₉)ₜ—W′—Ar′, or NH—W′—Ar′, t being an integer between 1 and 10, preferably between 1 and 4;
R₂₉ represents a group selected from (C₂-C₃₆)alkylene;
W′ represents a single bond, —CONR₃₀—, or —NR₃₁CO—;
Ar′ represents a (C₅-C₃₆)aryl group, said group being substituted with one, two or three atoms of iodine and/or bromine, and optionally substituted with one to four, preferably two or three, groups selected from (C₁-C₁₀)alkyl, —NR₃₂R₃₃, —NR₃₄COR₃₅, —COOR₃₆, —OR₃₇, —OCOR₃₈, —CONR₃₉R₄₀, —OCONR₄₁R₄₂, —NR₄₃COOR₄₄, NR₄₅CONR₄₆R₄₇, —OCOOR₄₈, and —COR₄₉;
R₃₀ and R₃₁ represent, independently of one another, a hydrogen atom or a (C₁-C₆)alkyl;
R₃₂ to R₄₉ represent, independently of one another, a hydrogen atom, a (C₁-C₁₀)alkyl, said (C₁-C₁₀)alkyl optionally being substituted with 1 to 10 OH groups, or a group —(CH₂—CH₂—O)_{t′}—R″, R″ being a hydrogen atom or a –(C₁-C₆)alkyl and t′ being an integer between 1 and 10, preferably between 1 and 5.

Advantageously, R₂₈ represents a (C₁-C₆)alkyl, more advantageously a (C₁-C₃)alkyl, more advantageously a methyl.

Advantageously, R₂₉ represents a (C₂-C₁₈)alkylene, more particularly a (C₂-C₆)alkylene, more advantageously an ethylene.

Advantageously, R₃₀ and R₃₁ represent, independently of one another, a hydrogen atom. Thus, W′ advantageously represents a single bond, —C(O)NH—, or —NHC(O)—.

Advantageously, Ar′ represents a (C₅-C₁₀)aryl, more particularly a phenyl, substituted with one, two or three atoms of iodine and/or bromine, preferably of iodine, and optionally two or three groups selected from (C₁-C₁₀)alkyl, —NR₃₂R₃₃, —NR₃₄C(O)R₃₅, —C(O)OR₃₆, —OR₃₇, —OC(O)R₃₈, —C(O)NR₃₉R₄₀, —OC(O)NR₄₁R₄₂, —NR₄₃C(O)OR₄₄, —NR₄₅C(O)NR₄₆R₄₇, —OC(O)OR₄₈, and —C(O)R₄₉.

Advantageously, Ar′ represents a (C₅-C₁₀)aryl, more particularly a phenyl, substituted with three atoms of iodine and/or of bromine, preferably of iodine, and optionally two groups selected from (C₁-C₁₀)alkyl, —NR₃₂R₃₃, —NR₃₄C(O)R₃₅, —C(O)OR₃₆, —OR₃₇, —OC(O)R₃₈, —C(O)NR₃₉R₄₀, —OC(O)NR₄₁R₄₂, —NR₄₃C(O)OR₄₄, —NR₄₅C(O)NR₄₆R₄₇, —OC(O)OR₄₈, and —C(O)R₄₉.

Advantageously, Ar′ represents a phenyl substituted with three atoms of iodine and/or of bromine, preferably of iodine, and optionally two groups selected from (C₁-C₁₀)alkyl, —NR₃₂R₃₃, —NR₃₄C(O)R₃₅, —C(O)OR₃₆, —OR₃₇, —OC(O)R₃₈, —C(O)NR₃₉R₄₀, —OC(O)NR₄₁R₄₂, —NR₄₃C(O)OR₄₄, —NR₄₅C(O)NR₄₆R₄₇, —OC(O)OR₄₈, and —C(O)R₄₉, advantageously from (C₁-C₁₀)alkyl, —NR₃₂R₃₃, —NR₃₄C(O)R₃₅, —C(O)OR₃₆, —OR₃₇, —C(O)NR₃₉R₄₈, —NR₄₃C(O)OR₄₄, NR₄₅C(O)NR₄₆R₄₇, —OC(O)OR₄₈, and —C(O)R₄₉.

Advantageously, the compound of general formula (V) is selected from the following compounds:

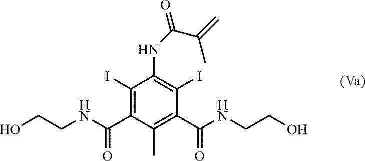

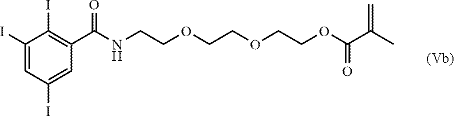

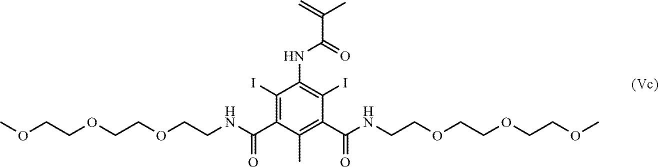

, and

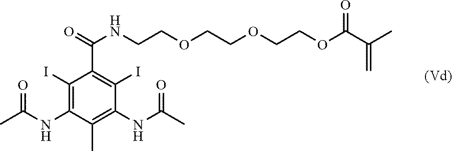 [SMILES: C=C(C)C(=O)OCCOCCOCCNC(=O)c1c(I)c(NC(C)=O)c(C)c(NC(C)=O)c1I]

In the context of the present invention, the compound of general formula (V) as defined above is advantageously used as a radiopaque halogenated monomer. Thus, the present invention also relates to the use of the compound of general formula (V) as defined above as a radiopaque halogenated monomer.

The examples given hereunder are intended to illustrate the present invention. Hereinafter, the word “microsphere”, whether in the singular or in the plural, will generally be abbreviated to “MS”.

### Examples

### Example 1a: Synthesis of a Tri-Iodinated Monomer, 2-Methacryloyloxyethyl (2,3,5-Triiodobenzoate) (MAOETIB)

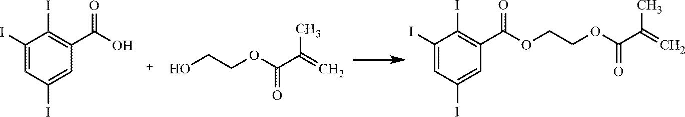

40 g (80 mmol) of 2,3,5-triiodobenzoic acid is added in small portions to a solution at 0° C. of diethyl ether (400 mL) containing 11.46 g (88 mmol, 1.1 eq.) of 2-hydroxyethyl methacrylate, 18.17 g (88 mmol, 1.1 eq.) of 1,3-dicyclohexylcarbodiimide and 1.19 g (8 mmol, 0.1 eq.) of 4-pyrrolidinopyridine. The solution is stirred for one hour at 0° C. and then for 18 h at 25° C. The solid that forms is filtered on a frit and washed with diethyl ether several times. The ether solution is then washed with hydrochloric acid solution (2 N) and then with a saturated solution of sodium bicarbonate. The organic phase is dried over magnesium sulfate. After filtration, the solvent is removed in a rotary evaporator to give an orange solid. The crude product is then purified on silica gel, eluting with a solution of petroleum ether/ethyl acetate (9/1). After evaporation of the solvent, an orange-tinted solid is obtained and is purified again by recrystallization by slow diffusion in a mixture of ethyl acetate in petroleum ether at 4° C.overnight. After filtration, washing with the cold solution and drying under vacuum, 31.1 g of pure white flakes of MAOETIB are obtained (yield = 64%). ¹H NMR (CDCl₃) 1.97 (s, 3H, CH₃), 4.57 and 4.48 (m, 4H, OCH₂CH₂O), 5.61 (s, 1H, olefinic), 6.16 (s, 1H, olefinic), 7.33 (d, 1H), 8.30 (d, 1H).

### Example 1b: Synthesis of a Tri-Iodinated Monomer, 2-(2-(2-(2,3,5-Triiodobenzamido)ethoxy)ethyl Methacrylate (Formula Vb)

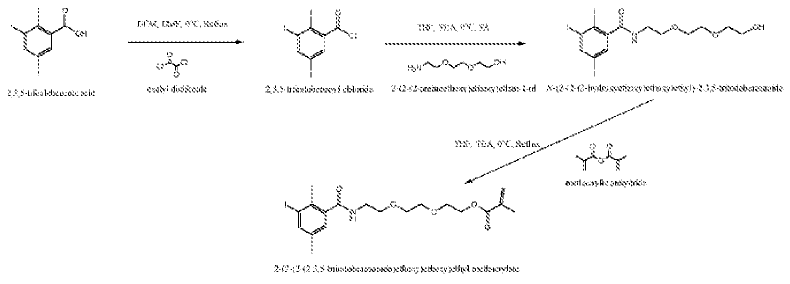

### Step 1

20.0 g (40.0 mmol) of 2,3,5-triiodobenzoic acid isdissolved in dichloromethane (60 mL), to which dimethylformamide is added (a few drops). The reaction mixture is then placed under argon and is cooled to a temperature of 0° C. 17.15 mL (200 mmol) of oxalyl chloride is then added dropwise over a period ranging from 5 to 10 min while maintaining the temperature of the reaction mixture close to 0° C. The solution is kept stirred until it is back at room temperature and is then heated under reflux (70° C.) for 30 h. The reaction mixture is then evaporated under vacuum. The solid obtained is co-evaporated with dichloromethane 3 to 4 times, so as to remove traces of oxalyl chloride still present. An orange-brown solid is obtained. The product is not isolated in this step, but is used directly in the rest of the synthesis.

### Step 2

13.44 g (90 mmol) of 2-[2-(2-aminoethoxy)ethoxy]ethan-1-ol is dissolved in 235 mL of anhydrous tetrahydrofuran (THF) at 60° C. The mixture is dried over MgSO₄, filtered and then poured into a three-necked flask. 12.6 mL (90.4 mmol) of triethylamine (TEA) is added to the mixture. The mixture is then placed under argon and then cooled to a temperature of 0° C. 20.74 g (40 mmol) of 2,3,5-triiodobenzoyl chloride is dissolved in 60 mL of anhydrous THF and added dropwise in the space of 5 minutes to the reaction mixture, maintaining a temperature close to 0° C. The solution is stirred for 4 hours at 0° C. and then overnight at room temperature (RT). The reaction mixture is then suspended in 1.8 L of water for one hour. The mixture is poured into a separating funnel, and 235 mL of dichloromethane (DCM) is added. The aqueous phase is washed 3 times with 115 mL of DCM. The organic phases are combined and then dried over MgSO₄. Vacuum evaporation is carried out until a brown oil is obtained. 23.4 g of this oil is obtained. The yield in this step is 92.7%.

### Step 3

23.4 g (37 mmol) of N- (2- (2- (2-hydroxyethoxy)ethoxy)ethyl)-2,3,5-triiodobenzamide is dissolved in 235 mL of anhydrous THF. 26 mL (186.5 mmol) of triethylamine is added to the mixture. The reaction mixture is cooled to T=0° C. 27.5 mL (185.5 mmol) of methacrylate anhydride is added dropwise to the mixture, keeping the temperature close to 0° C. The mixture is stirred for 3 hours at 0° C., and then under reflux (80° C.) overnight. The reaction mixture is then suspended in 1.5 L of water for one hour, and then decanted. 350 mL of DCM is added and the aqueous phase is washed 3 times with DCM (120 mL). The organic phases are combined and then dried over MgSO₄. After evaporation under vacuum, 32.64 g of a brown oil is recovered. The crude product is then purified by puriFlash®, on a silica column (330 g, Si40-60), with a DCM/acetonitrile (9/1) elution mixture. After evaporation of the solvent, 11.31 g of a white solid is obtained.

The total yield is 40.4%.

Conditions of the HPLC-MS method of analysis:
BEH C18 column No. 516
T_{furnace} = 30° C.
Composition of mobile phase: Water-HCO₂H 0.5% (v/v) / MeCN
Isocratic gradient 55/45
Flow 0.7 mL/min
Volume injected = 1 µL
λ = 235 nm

Results:
Retention time: 2.2 min
Mass m/z: 699.89
Purity UV: 82.3%

¹H NMR (Acetone) 1.97 (s, 3H, CH₃), 2.93 (t, 2H, NCH₂), 3.57 (m, 10H, CH₂OCH₂CH₂OCH₂), 4.32 (t, 2H, CH₂O), 5.65 (s, 1H, olefinic), 6.15 (s, 1H, olefinic), 7.65 (d, 2H, benzyl and NH), 8.38 (d, 1H, benzyl).

### Example 2

Synthesis by Direct Suspension polymerization of polymers containing MAOETIB according to the invention in the form of microspheres with a size of 700-900 µm with variation of the concentration of monomers

An aqueous solution of hydrolyzed polyvinyl alcohol and sodium chloride is poured into a reactor and is heated to 50° C. The organic phase containing poly(ethylene glycol) methyl ether methacrylate (m-PEGMA) (hydrophilic monomer), poly(ethylene glycol) dimethacrylate (PEGDMA) (crosslinking agent), methacrylic acid (MA) (ionizable monomer), MAOETIB (radiopaque monomer), hexanethiol (transfer agent), (1- (4- ( (2-methacryloxyethyl)oxy)phenylamino)-anthraquinone) violet dye and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved.

Table 1 below summarizes the main parameters and the composition of the organic phase.

TABLE 1

| | | | |
|---|---|---|---|
| | | | VizBeads 700-900 µm Batches 4, 5 and 6 |
| Process parameters | O/W (oil/water) volume ratio | | ⅙ |
| | Total volume | | 980 mL |
| | Volume of organic phase | | 140 mL |
| | Stirring speed | | 100 rpm |
| | PVA (30-70 kDa) | | 0.25% (by weight relative to the aqueous phase) |
| | NaCl | | 7% (by weight relative to the aqueous phase) |
| Organic phase | Weight of monomer / weight of the organic phase (%) | | 35% (batch 6), 38% (batch 5) or 40% (batch 4) by weight of the organic phase |
| | Hexanethiol | | 3 mol% / mol of m-PEGMA |
| | AIBN | | 1 mol% / mol of methacrylate function |
| | Monomer phase | m-PEGMA | 64.96 mol%/total moles of monomers |
| | | PEGDMA | 5 mol%/total moles of monomers |
| | | MA | 10 mol%/total moles of monomers |
| | | MAOETIB | 20 mol%/total moles of monomers |
| | | Dye | 0.04 mol%/ total moles of monomers |

### Example 3

Synthesis by Direct Suspension polymerization of polymers containing different concentrations of MAOETIB according to the invention in the form of microspheres

An aqueous solution of hydrolyzed polyvinyl alcohol and sodium chloride is poured into a reactor and is heated to 50° C. The organic phase containing poly(ethylene glycol) methyl ether methacrylate (m-PEGMA) (hydrophilic monomer), poly(ethylene glycol) dimethacrylate (PEGDMA) (crosslinking agent), methacrylic acid (MA) (ionizable monomer), MAOETIB (radiopaque monomer), hexanethiol (transfer agent), (1- (4- ( (2-methacryloxyethyl)oxy)phenylamino)-anthraquinone) violet dye and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved.

Table 2 below summarizes the main parameters and the composition of the organic phase.

TABLE 2

| | | | 100-300 µm Batch 13 | 500-700 µm Batches 16 and 19 | 700-900 µm Batch L6 |
|---|---|---|---|---|---|
| Process parameters | O/W (oil/water) volume ratio | | 1/11 | ⅙ | ⅙ |
| | Total volume | | 460 mL | 460 mL | 490 mL |
| | Volume of organic phase | | 38 mL | 66 mL | 70 mL |
| | Stirring speed | | 180 rpm | 100 rpm | 100 rpm |
| | PVA (by weight relative to the aqueous phase) | | 13-23 kDa 0.5% | 30-70 kDa 0.25% | 30-70 kDa 0.25% |
| | NaCl (by weight relative to the aqueous phase) | | 3% | 7% | 7% |
| Organic phase | Weight of monomer / weight of the organic phase (%) | | 56% | 32% | 35% |
| | monomers | m-PEGMA in mol/total mol of monomers | 44.96% | 64.96% (batch 19) 54.96% (batch 16) | 19.96% |
| | | PEGDMA in mol/total mol of monomers | 5% | 5% | 5% |
| | | MA in mol/total mol of monomers | 30% | 10% | 0% |
| | | MAOETIB in mol/total mol of monomers | 20% | 20% (batch 19) 30% (batch 16) | 75% |
| | | Dye in mol/total mol of monomers | 0.04% | 0.04% | 0.04% |
| | Transfer agent | Hexanethiol in mol/mol of m-PEGMA | 3% | 3% | 3% |
| | Initiator | AIBN in mol/mol of methacrylate function | 1% | 1% | 1% |

### Example 4

Synthesis by Direct Suspension polymerization of polymers containing USPIO according to the invention in the form of microspheres

An aqueous solution of hydrolyzed polyvinyl alcohol and sodium chloride is poured into a reactor and is heated to 50° C. The organic phase containing poly(ethylene glycol) methyl ether methacrylate (m-PEGMA) (hydrophilic monomer), poly(ethylene glycol) dimethacrylate (PEGDMA) (crosslinking agent), methacrylic acid (MA) (ionizable monomer), MAOETIB (radiopaque monomer), hexanethiol (transfer agent), (1- (4- ( (2-methacryloxyethyl)oxy)phenylamino)-anthraquinone) violet dye, USPIO and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved. Table 3 below summarizes the main parameters and the composition of the organic phase.

TABLE 3

| | | | 100-300 µm Batches 21 to 26 |
|---|---|---|---|
| Process parameters | O/W (oil/water) volume ratio | | 1/11 |
| | Total volume | | 460 mL |
| | Volume of organic phase | | 38 mL |
| | Stirring speed | | 180 rpm |
| | PVA (13-23 kDa) (by weight relative to the aqueous phase) | | 1% |
| | NaCl (by weight relative to the aqueous phase) | | 3% |
| Organic phase | Weight of monomer/weight of the organic phase (%) | | 56% |
| | monomers | m-PEGMA in mol/total mol of monomers | 44.96% |
| | | PEGDMA in mol/total mol of monomers | 5% |
| | | MA in mol/total mol of monomers | 30% |
| | | MAOETIB in mol/total mol of monomers | 20% |
| | | Dye in mol/total mol of monomers | 0.04% |
| | Particles visible in MRI | USPIO (10, 20 or 30 nm) By volume relative to the organic phase | 0.1% or 0.5% or 1% |
| | Transfer agent | Hexanethiol in mol/mol of PEGma | 3% |
| | Initiator | AIBN in mol/mol of function methacrylate | 1% |

### Example 5

Synthesis by Direct Suspension polymerization of Polymers According to the Invention containing MAOETIB and not comprising methacrylic acid (MA), in the form of microspheres with a size of 300-500 µm and 700-900 µm

An aqueous solution of hydrolyzed polyvinyl alcohol and sodium chloride is poured into a reactor and is heated to 50° C. The organic phase containing poly(ethylene glycol) methyl ether methacrylate (m-PEGMA) (hydrophilic monomer), poly(ethylene glycol) dimethacrylate (PEGDMA) (crosslinking agent), MAOETIB (radiopaque monomer), hexanethiol (transfer agent), (1- (4- ( (2-methacryloxyethyl)oxy)phenylamino)-anthraquinone) violet dye and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved. Two fractions are recovered, microspheres of size 300-500 µm and microspheres of size 500-700 µm.

Table 4 below summarizes the main parameters and the composition of the organic phase.

TABLE 4

| | | | | |
|---|---|---|---|---|
| | | | Microspheres 300-500 µm Batch L1 | Microspheres 500-700 µm Batch L1 Bis |
| Process parameters | O/W (oil/water) volume ratio | | ⅙ | ⅙ |
| | Total volume | | 550 mL | 550 mL |
| | Volume of organic phase | | 79 mL | 79 mL |
| | Stirring speed | | 105 rpm | 105 rpm |
| | PVA (30-70 kDa) | | 0.25% (by weight relative to the aqueous phase) | 0.25% (by weight relative to the aqueous phase) |
| | NaCl | | 7% (by weight relative to the aqueous phase) | 7% (by weight relative to the aqueous phase) |
| Organic phase | Weight of monomer/ weight of the organic phase (%) | | 35% by weight of the organic phase | 35% by weight of the organic phase |
| | Hexanethiol | | 3 mol% / mol of m-PEGMA | 3 mol% / mol of m-PEGMA |
| | AIBN | | 1 mol% / mol of methacrylate function | 1 mol% / mol of methacrylate function |
| | Monomer phase | m-PEGMA | 74.96 mol%/total moles of monomers | 74.96 mol%/moles total of monomers |
| | | PEGDMA | 5 mol%/total moles of monomers | 5 mol%/total moles of monomers |
| | | MA | 0 mol%/total moles of monomers | 0 mol%/total moles of monomers |
| | | MAOETIB | 20 mol%/total moles of monomers | 20 mol%/total moles of monomers |
| | | Dye | 0.04 mol%/ total moles of monomers | 0.04 mol%/ total moles of monomers |

### Characterizations

The dry extract (dry weight) is determined as follows: 1 ml of sedimented MS is placed in a 5 ml Eppendorf vial, frozen at -80° C. and lyophilized in a lyophilizer (Heto PowerDry® LL 1500, Thermo Scientific) overnight. The mass of the microspheres after lyophilization is then measured. Measurement was carried out for three samples and the mean value was taken as the final value of the dry matter of the MS.

The average diameter is measured by analyzing microscopy images of 2000 microspheres (Morphologi 4, Malvern).

The test of injectability in microcatheters is carried out with 1 mL of sediment of microspheres suspended beforehand in 10 mL of iodinated contrast medium (70% of Optiray® 300, Guerbet, 30% of saline solution). A homogeneous suspension of microspheres in a 3 mL syringe is then injected in the microcatheter. The microcatheters, which are supplied by the Terumo company, were selected so that their inside diameter is just slightly greater than the average diameter of the microspheres. The resistance felt during injection of the microspheres in the microcatheter is recorded (Table 4Bis). Blockage during injection would signify failure of injection. After injection, the microspheres are observed with the microscope in order to check whether the microspheres regain their spherical shape.

Results:

TABLE 4Bis

| Batches | Size | Dry weight per mL of wet sediment (mg/mL) | Average diameter (µm) | Injectability |
|---|---|---|---|---|
| Batch L1 | 300-500 | 185 | 403 ± 39 | Progreat®2.0 Fr (ID(1) = 490 µm): no blockage, very slight resistance |
| Batch L1bis | 500-700 | 149 | 615 ± 46 | Progreat®2.7 Fr (ID(1) = 650 µm): no blockage, very slight resistance |

### Example 6

Other Syntheses by Direct Suspension polymerization of polymers according to the invention in the form of microspheres

An aqueous solution of hydrolyzed polyvinyl alcohol and sodium chloride is poured into a reactor and is heated to 50° C. The organic phase containing the main hydrophilic monomer, crosslinking agent, radiopaque monomer, optionally ionizable monomer, transfer agent, (1-(4-((2-methacryloxyethyl)oxy)phenylamino)-anthraquinone) violet dye and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved.

Table 5 below summarizes the main parameters and the composition of the organic phase.

TABLE 5

| | | | | |
|---|---|---|---|---|
| | | | Microspheres 700-900 µm Batch L2 | Microspheres 500-700 µm Batch L4 |
| Process parameters | O/W (oil/water) volume ratio | | ⅙ | ⅙ |
| | Total volume | | 490 mL | 490 mL |
| | Volume of organic phase | | 60 mL | 60 mL |
| | Stirring speed | | 105 rpm | 105 rpm |
| | PVA (30-70 kDa) | | 0.25% (by weight relative to the aqueous phase) | 0.25% (by weight relative to the aqueous phase) |
| | NaCl | | 7% (by weight relative to the aqueous phase) | 7% (by weight relative to the aqueous phase) |
| Organic phase | Weight of monomer/ weight of organic phase (%) | | 35% by weight of the organic phase | 35% by weight of the organic phase |
| | Transfer agent | | Hexanethiol 3 mol%/ mol of hydrophilic monomer | Bromotrichloromethane 3 mol%/mol of hydrophilic monomer |
| | AIBN | | 1 mol%/mol of methacrylate function | 1 mol%/mol of methacrylate function |
| | Monomer phase | Hydrophilic monomer | N-vinylpyrrolidone 64.96 mol%/total moles of monomers | PEGMA 64.96 mol%/moles total of monomers |
| | | Crosslinking agent | PEG diacrylate (700 Da) 5 mol%/total moles of monomers | PEGDMA 5 mol%/total moles of monomers |
| | | Ionizable monomer | Methacrylic acid 10 mol%/total moles of monomers | Methacrylic acid 10 mol%/total moles of monomers |
| | | Radiopaque monomer | MAOETIB 20 mol%/total moles of monomers | Compound from Example 1b) (of formula (Vb)) 20 mol%/total moles of monomers |
| | | Dye | 0.04 mol%/total moles of monomers | 0.04 mol%/total moles of monomers |

The average diameters of the microspheres of batches L2 and L4 are 731 ±53 and 652 ±39, respectively.

### Example 7

Synthesis by Direct Suspension polymerization of polymers containing different concentrations of transfer agent according to the invention in the form of microspheres

An aqueous solution of hydrolyzed polyvinyl alcohol and sodium chloride is poured into a reactor and is heated to 50° C. The organic phase containing poly(ethylene glycol) methyl ether methacrylate (m-PEGMA) (hydrophilic monomer), poly(ethylene glycol) dimethacrylate (PEGDMA) (crosslinking agent), methacrylic acid (MA) (ionizable monomer), MAOETIB (radiopaque monomer), hexanethiol (transfer agent), (1- (4- ( (2-methacryloxyethyl)oxy)phenylamino)-anthraquinone) violet dye and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved.

Table 6 below summarizes the main parameters and the composition of the organic phase.

TABLE 6

| | | | 700-900 µm Batch L5 |
|---|---|---|---|
| Process parameters | O/W (oil/water) volume ratio | | ⅙ |
| | Total volume | | 490 mL |
| | Volume of organic phase | | 70 mL |
| | Stirring speed | | 105 rpm |
| | PVA (by weight relative to the aqueous phase) | | 30-70 kDa 0.25% |
| | NaCl (by weight relative to the aqueous phase) | | 7% |
| Organic phase | Weight of monomer/weight of the organic phase (%) | | 35% |
| | monomers | m-PEGMA in mol/total mol of monomers | 74.96% |
| | | PEGDMA in mol/total mol of monomers | 5% |
| | | MA in mol/total mol of monomers | 10% |
| | | MAOETIB in mol/total mol of monomers | 20% |
| | | Dye in mol/total mol of monomers | 0.04% |
| | Transfer agent | Hexanethiol in mol/mol of m-PEGMA | 15% |
| | Initiator | AIBN in mol/mol of methacrylate function | 1% |

### Characterizations

Characterization is carried out in the same way as in example 5 and the results are presented in Table 6bis.

Results:

TABLE 6bis

| Batches | Transfer agent | Ratio of weight of monomer /weight of the organic phase (%) | MAOETI B (%) | MA (%) | Dry weight per ml of wet sediment (mg/mL) | Average diameter (µm) | Injectability in a Progreat® 2.8 microcatheter (ID(1) = 700 µm) |
|---|---|---|---|---|---|---|---|
| Batch 3 | 0 | 35 | 20 | 10 | 231 | 803 | Blockage |
| Batch 6 | 3% | 35 | 20 | 10 | 135 | 895 | No blockage. Low resistance |
| Batch L5 | 15% | 35 | 20 | 10 | No microspheres formed | | |

These results thus demonstrate the effect of adding a transfer agent on the injectability of the microspheres and the advantages of the concentration range selected. An amount of transfer agent well above this range does not allow microspheres to be obtained.

### Example 8

Synthesis by Direct Suspension polymerization of polymers containing the compound from example 1b) (of formula (Vb)) as radiopaque halogenated monomer according to the invention in the form of microspheres

An aqueous solution of hydrolyzed polyvinyl alcohol and sodium chloride is poured into a reactor and heated to 50° C. The organic phase containing poly(ethylene glycol) methyl ether methacrylate (m-PEGMA) (hydrophilic monomer), poly(ethylene glycol) dimethacrylate (PEGDMA) (crosslinking agent), methacrylic acid (MA) (ionizable monomer), the compound from example 1b) (radiopaque monomer), bromotrichloromethane (transfer agent), (1-(4-((2-methacryloxyethyl)oxy)phenylamino)-anthraquinone) violet dye and AIBN (initiator) dissolved in toluene is then fed into the reactor. Stirring is applied with a stirrer of the propeller type at a suitable speed for obtaining droplets with the desired diameter. The temperature is then increased to 80° C. and stirring is maintained for 8 hours. The mixture is then filtered and the microspheres are washed with acetone and then with water before being sieved and then autoclaved.

Table 7 below summarizes the main parameters and the composition of the organic phase.

TABLE 7

| | | | 500-700 µm Batch L4 |
|---|---|---|---|
| Process parameters | O/W (oil/water) volume ratio | | ⅙ |
| | Total volume | | 490 mL |
| | Volume of organic phase | | 70 mL |
| | Stirring speed | | 105 rpm |
| | PVA (by weight relative to the aqueous phase) | | 30-70 kDa |
| | | | 0.25% |
| | NaCl (by weight relative to the aqueous phase) | | 7% |
| Organic phase | Weight of monomer/weight of the organic phase (%) | | 35% |
| | monomers | m-PEGMA in mol/total mol of monomers | 64.96% |
| | | PEGDMA in mol/total mol of monomers | 5% |
| | | MA in mol/total mol of monomers | 10% |
| | | Molecule from example 1b) in mol/total mol of monomers | 20% |
| | | Dye in mol/total mol of monomers | 0.04% |
| | Transfer agent | Bromotrichloromethane in mol/mol of m-PEGMA | 3% |
| | Initiator | AIBN in mol/mol of methacrylate function | 1% |

### Example 9

Effect of the Transfer Agent on The injectability of microspheres of 700-900 µm comprising polymers according to the invention in a microcatheter

The microspheres are prepared as indicated in example 2 for batches 4, 5, 6 and L6. Batches 1, 2 and 3 are synthesized equivalently but without adding transfer agent. The injectability in a microcatheter (Progreat® 2.8 Fr, Terumo, inside diameter 700 µm) is performed on 1 mL of sediment of microspheres suspended beforehand in 10 mL of iodinated contrast medium (70% of Iopamiron® 300, Bracco, 30% of saline solution or for batch L6: 70% of Optiray® 300, Guerbet, 30% of saline solution). A homogeneous suspension of microspheres in a 3 mL syringe is then injected in the microcatheter. The average diameter of the microspheres was selected greater than the inside diameter of the catheter, so as to demonstrate the flexibility of the microspheres. The resistance felt during injection of the microspheres in the microcatheter is recorded (Table 8). Blockage during injection signifies injection failure. After injection, the microspheres are observed with the microscope in order to check whether the microspheres regain their spherical shape.

TABLE 8

Injectability of microspheres 700-900 µm comprising polymers according to the invention in a microcatheter

| Batches | Transfer agent | Ratio (%) weight of monomer/ weight of the organic phase | % MAOETIB | Dry weight per mL of wet sediment (mg/mL) | Average diameter (µm) | Injectability in PG2.8 (ID⁽¹⁾ = 700 µm) |
|---|---|---|---|---|---|---|
| Batch 1 | No | 40 | 20 | 231-235 | 770 | Blockage |
| Batch 2 | No | 38 | 20 | 236 | 781 | Blockage |
| Batch 3 | No | 35 | 20 | 233 | 831 | Blockage |
| Batch 4 | Yes | 40 | 20 | 155 | 857 | No blockage. Low resistance |
| Batch 5 | Yes | 38 | 20 | 158 | 827 | No blockage. Low resistance |
| Batch 6 | Yes | 35 | 20 | 135 | 895 | No blockage. Low resistance |
| Batch L6 (without MA) | Yes | 35 | 75 | 831 | 293^{∗} | Not injectable^{∗∗} |

⁽¹⁾ ID = inside diameter of the microcatheter
* Owing to the high proportion of MAOETIB, the MS gel is too hydrophobic to swell and reach the desired size.
** The microspheres are sticky and they aggregate, preventing proper injection.

After injection, the microspheres prepared with the transfer agent according to the invention maintain their spherical shape and are not broken.

In the absence of transfer agent, the microspheres block the microcatheter.

In the presence of the transfer agent, the microspheres comprising polymers according to the invention are easily injectable, i.e. they only offer low resistance to injection and do not block the microcatheter.

### Example 10

Visibility of the Microspheres According to the invention to X-rays in vivo

The visibility of the microspheres according to example 3 implanted subcutaneously in the rabbit is analyzed 3 months after implantation. The animals (n = 2) are euthanized, the back is shaved and a 26 G needle is placed in the skin at the injection site of the microspheres to serve as reference. A fluoroscopic/radiographic mobile unit (GE Healthcare – OEC 9900 Elite) is used for taking photographs of the animals’ backs (X-ray beam energy of 63 kV, current intensity 1.3 mA). Quantification of radio-opacity in Hounsfield units (HU) was carried out using ANALYZE 11.0 software (Table 9).

TABLE 9

X-ray imaging of the microspheres injected in the skin of the rabbit

| Batches | Diameter (µm) | Radio-opacity | Number of Hounsfield units (HU) | Visibility of the microspheres (MS) by comparing with the radio-opacity of the nearest rib |
|---|---|---|---|---|
| Embosphere® | 500-700 | No | NA | Not visible |
| Batch 13 | 100-300 | Yes | 1950 | Equal visibility between the MS and the rib |
| Batch 16 | 500-700 | Yes | 2200 | Equal visibility between the MS and the rib |
| Batch 19 | 500-700 | Yes | 1500 | Visibility lower than the radio-opacity of the rib |

The radiopaque microspheres implanted in the dermis of the skin of the rabbit are visible to X-rays (Table 9). The intensity of the microspheres is close to that observed for the animals’ ribs. The microspheres without iodine (Embosphere®) are not visible to X-rays.

### Example 11

Loading and Release of Active ingredients on the radiopaque microspheres according to the invention

The tests of loading and controlled release of anticancer drugs were performed on radiopaque microspheres of 100-300 µm sterilized by autoclaving and comprising or not comprising an ionized or ionizable monomer such as methacrylic acid.

The microspheres with methacrylic acid are microspheres from batch 13, the composition of which is given in example 3. The microspheres without methacrylic acid have the same composition as the microspheres from batches L1 and L1Bis described in example 5.

Loading with doxorubicin: the target for loading is 37.5 mg of doxorubicin per ml of microspheres. For this, 3.8 mL of doxorubicin-HCl (Adriblastine®, Pfizer) in solution in water at 2.5 mg/mL is added to 250 µL of wet sediment of microspheres. After mixing by inversion, the suspension is made up to 6 mM with sodium bicarbonate (Lavoisier). Loading is carried out at room temperature and with stirring for one hour. Measurement of the residual amount of doxorubicin (absorbance at 490 nm) present in the supernatants serves for determining the amount of drug loaded on the microspheres.

To study the release of doxorubicin from the microspheres, the sediments are washed in 10 mL of water, before adding 50 mL of buffer 50 mM Tris-HCl, 0.9% NaCl, pH 7.4. Incubation takes place at 37° C. with stirring. The release of doxorubicin is measured at different times at 490 nm.

Loading with irinotecan: the loading target is 50 mg of irinotecan per mL of microspheres. The sediments of the radiopaque microspheres are incubated for 30 minutes in an excess of sodium bicarbonate (1.4%, Lavoisier) without stirring. Then the supernatant is removed, and 625 µL of irinotecan solution at 20 mg/mL (Campto, Pfizer) is added. After 30 minutes, measurement of the residual amount of irinotecan (absorbance at 370 nm) in the supernatant serves for determining the amount loaded on the microspheres.

To study the release of irinotecan, the microspheres are washed in 10 mL of water, and then 50 mL of PBS (10 mM Na₂HPO₄, 1.8 mM KH₂PO₄, 138 mM NaCl, 2.7 mM KCl, pH 7.4) equilibrated at 37° C. is added. The release of the drug over time is measured by reading the absorbance at 370 nm.

Loading with sunitinib: The sediments of the radiopaque microspheres are incubated for 1 h at room temperature in 10 mL of sunitinib in the form of malate (LC Laboratories) at 1 mg/mL in water. The final concentration of sodium bicarbonate is 4 mM. After stirring for 1 h on a wheel, measurement of the residual amount of sunitinib (absorbance at 405 nm) in the supernatant serves for determining the amount loaded on the microspheres.

To study the release of sunitinib, the microspheres are washed in 10 mL of water, and then 50 mL of PBS equilibrated at 37° C. is added. The release of the drug over time is measured by reading the absorbance at 405 nm.

Loading with vandetanib: The sediments of the radiopaque microspheres are incubated for 2 h at room temperature in 10 mL of a water/DMSO (1/1) mixture containing 5 mg of vandetanib (LC Laboratories). The residual amount of vandetanib in the supernatants is measured at 254 nm in order to calculate the amount loaded on the microspheres.

To study the release of vandetanib, the microspheres are washed in 10 mL of water, and then 50 mL of PBS equilibrated at 37° C. is added. The release of the drug over time is measured by reading the absorbance at 254 nm.

TABLE 10

Loading of anticancer drugs on the radiopaque microspheres and release thereof in vitro

| | | | | Release in vitro (37° C., 150 rpm) | |
|---|---|---|---|---|---|
| | Anticancer drugs | Loading (mg of drug / mL of wet sediment) | % elution at 1 h | Time for 50% release | Time for complete release |
| Without methacrylic acid | Doxorubicin | 29.8 | ND | ND | ND |
| With methacrylic acid | Doxorubicin | 36.8 | 26 | 1 day | 1 week |
| | Irinotecan | 46 | 50 | 1 h | 1 day |
| | Sunitinib | 37 | 12 | 6 days | > 15 days |
| | Vandetanib | 11 | 17 | 2 days | 15 days |

Loading of various anticancer drugs (cytotoxic and antiangiogenic drugs) is possible on the radiopaque microspheres of diameter 100-300 µm. Loading of the drugs on the microspheres is quick (less than 2 h).

Elution in PBS depends on the drugs loaded. Release of irinotecan is rapid (50% in 1 h) ; release of doxorubicin, sunitinib and vandetanib is slower, being spread over several days.

Loading efficiency is calculated with the following equation:

$$L C \left(m g o f d r u g / m L o f M S\right) = \frac{\begin{matrix}{m}_{D r u g i n i t i a l} - {C}_{D r u g \_ sup} \\ ∗ {V}_{sup}\end{matrix}}{{V}_{M S}}$$

$$L E ( \% ) = \frac{L C}{{{m}_{D r u g}}_{i n i t i a l}}$$

LC: Loading capacity
LE: Loading efficiency
M_{Drug}ᵢₙᵢₜᵢₐₗ: Amount of drug dissolved
C_{Drug_sup}: Concentration of the drug in the supernatant after loading
Vₛᵤₚ: Volume of the supernatant
V_{MS}: Volume of microspheres

The loading efficiency without methacrylic acid is 83.5%, compared to 99.7% in the presence of 20% of methacrylic acid. The studies carried out show that the efficiency of loading is lower for the microspheres without methacrylic acid than for those comprising methacrylic acid.

The capacity of the microspheres without ionizable monomer for loading doxorubicin is explained by the establishment of hydrophobic or van der Waals bonds. In the presence of ionizable monomer, besides these bonds, doxorubicin is loaded by electrostatic bonds. The kinetics and the loading capacities are improved thereby.

### Example 12

Modification of the Signal in MRI In vitro of the microspheres according to the invention loaded with USPIO: measurement of T2

The microspheres according to example 4 were suspended in 2% agarose gel 50/50 v/v. The microsphere inserts were embedded in a plate of agarose gel at 2%. The plate was imaged using a 1.5 T MRI (Phillips). The sequence used for this imaging is as follows: Sequence T2: TR = 2000 ms, TE from 10 ms to 310 ms steps of 20 ms. Voxel = 0.5*0.5*2 mm, treatment under Matlab to obtain T2. The size of the voxels is 0.5*0.5*1 mm. The FOV (Field of View) is 150*150 mm.

TABLE 11a

comparison of microspheres loaded with increasing amounts of USPIOs

| | | |
|---|---|---|
| Batch 21 100-300 µm 0.1% USPIOs 30 nm | Batch 22 100-300 µm 0.5% USPIOs 30 nm | Batch 23 100-300 µm 1% USPIOs 30 nm |
| T2 =130 ms | T2 =111.7 ms | T2 =97.06 ms |

TABLE 11b

comparison of microspheres loaded with USPIOs of 10, 20 or 30 nm

| | | |
|---|---|---|
| Batch 24 100-300 µm 1% USPIOs 10 nm | Batch 25 100-300 µm 1% USPIOs 20 nm | Batch 26 100-300 µm 1% USPIOs 30 nm |
| T2 =77.4 ms | T2 =94.94 ms | T2 =97.06 ms |

### Table 11. MRI Measurement of T2 of the Microspheres

The drop in signal intensity is in agreement with the T2 effect of the USPIOs. The signal intensity increases with the amount of USPIO of the microspheres. It also increases with the decrease in size of the USPIOs (from 30 nm to 10 nm).

1. A polymer comprising a crosslinked matrix, said matrix being based on at least:
a) 20% to 90% of hydrophilic monomer selected from N-vinylpyrrolidone, and a monomer of the following formula (I): 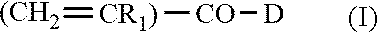
in which:
D represents O—Z or NH—Z, Z representing (C₁-C₆)alkyl, —(CR₂R₃)ₘ—CH₃, —(CH₂—CH₂—O)ₘ—H, —(CH₂—CH₂—O)ₘ—CH₃, —C(R₄OH)ₘ or —(CH₂)m—NR₅R₆ with m representing an integer from 1 to 30;
R₁, R₂, R₃, R₄, R₅ and R₆ represent, independently of one another, H or a (C₁-C₆)alkyl;
b) 5% to 50% of halogenated radiopaque monomer of the following general formula (II):
(CH₂═CR₇)—CO—Y (II)
in which
Y represents O—W, (O—R₈)ₚ—W, (NH—Rs)p—W or NH—W, W representing Ar, L—Ar, and p being an integer between 1 and 10, in which:
Ar represents a (C₅-C₃₆)aryl or (C₅-C₃₆)heteroaryl group, said group being substituted with one, two or three atoms of iodine and/or bromine, and optionally substituted with one to four groups selected from (C₁-C₁₀)alkyl, —NRₐR_{b}, —NR_{c}COR_{d}, —COORₑ, —OR_{f}, —OCOR_{g}, —CONRₕᵢ, —OCONRⱼRₖ, —NR₁COOR₀—, —NRᵣCONR₅Rₜ, —OCOORᵤ, and —CORᵥ;
L represents —(CH₂)ₙ—, —(HCCH)ₙ—, -O-, -S-, —SO—, —SO₂—, —OSO_{2⁻}. —NR₉—, —CO—, —COO—, —OCO—, —OCOO—, —CONR₁₀—, —NR₁₁CO—, —OCONR₁₂—, —NR₁₃COO— or —NR₁₄CONR₁₅—, n being an integer from 1 to 10;
R₉ to R₁₅ and Rₐ to Rᵥ represent, independently of one another, a hydrogen atom, a (C₁-C₁₀)alkyl, said (C₁-C₁₀)alkyl optionally being substituted with 1 to 10 OH groups, or a group —(CH₂—CH₂—O)_{q}—R′, R′ being a hydrogen atom or a -(C₁-C₆)alkyl and q being an integer between 1 and 10;
R₇ represents H or a (C₁-C₆)alkyl;
Rs represents a group selected from (C₁-C₃₆)alkylene, (C₃-C36)cycloalkylene, (C₂-C₃₆)alkenylene, (C₃-C₃₆)cycloalkenylene, (C₂-C₃₆)alkynylene, (C₃-C₃₆)cycloalkynylene, (C₅-C₃₆)arylene and (C₅-C₃₆)heteroarylene,
c) 1% to 15% of nonbiodegradable linear or branched hydrophilic crosslinking agent having groups (CH₂═(CR₁₆))— at each of its ends, each R₁₆; independently representing H or a (C₁-C₆)alkyl; and
d) 0.1% to 10% of transfer agent selected from alkyl halides and cycloaliphatic or aliphatic thiols, and optionally having another functional group selected from the amino, hydroxy and carboxy groups,
the percentages of the monomers a) to c) being given in moles relative to the total number of moles of monomers and the percentages of compound d) being given in moles relative to the number of moles of the hydrophilic monomer a).

2. The polymer of claim 1, wherein the matrix is based on halogenated radiopaque monomer of general formula (II) in an amount greater than 7% and less than or equal to 50% (mol%), relative to the total number of moles of monomers.

3. The polymer of claim 1, wherein the hydrophilic monomer a) is selected from the group consisting of N-vinylpyrrolidone, vinyl alcohol, 2-hydroxyethylmethacrylate, sec-butyl acrylate, n-butyl acrylate, t-butyl acrylate, t-butyl methacrylate, methylmethacrylate, N-dimethylaminoethyl(methyl)acrylate, N,N-dimethylaminopropyl-(meth)acrylate, t-butylaminoethyl(methyl)acrylate, N,N-diethylaminoacrylate, poly(ethylene oxide) (meth)acrylate, methoxy poly(ethylene oxide) (meth)acrylate, butoxy poly(ethylene oxide) (meth)acrylate, poly(ethylene glycol) (meth)acrylate, methoxy poly(ethylene glycol) (meth)acrylate, butoxy poly(ethylene glycol) (meth)acrylate, poly(ethylene glycol) methyl ether methacrylate and mixtures thereof.

4. The polymer of claim 1, wherein the radiopaque monomer is of general formula (II), in which Y represents O—C₆H₄I, O—C₆H₃I₂, O—C₆H₂I₃, NH—C₆H₄I, NH—C₆H₃I₂, NH—C₆H₂I₃, O—CH₂—CH₂—C(O)—C₆H₄I, O—CH₂—CH₂—O—C(O)—C₆H₃I₂, O—CH₂—CH₂—O—C(O)—C₆H₂I₃, NH—CH₂—CH₂—C(O)—C₆H₄I, NH—CH₂—CH₂—O—C(O)—C₆H₃I₂, NH—CH₂—CH₂—O—C(O)—C₆H₂I₃.

5. The polymer of claim 1, wherein the radiopaque monomer is (tri-iodobenzoyl)oxo ethyl methacrylate of the following formula (IIa): 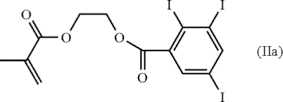
.

6. The polymer of claim 1, wherein the linear or branched, nonbiodegradable, hydrophilic crosslinking agent has groups (CH₂═(C₁₆))CO— or (CH₂═(CR₁₆))CO—O— at its at least two ends, each R₁₆ independently representing H or a (C₁-C₆)alkyl.

7. The polymer of claim 1, wherein the transfer agent is selected from thioglycolic acid, 2-mercaptoethanol, dodecanethiol, hexanethiol and mixtures thereof.

8. The polymer of claim 1, wherein the matrix is further based on at least one ionized or ionizable monomer of the following formula (IV): 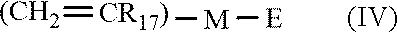
in which:
R₁₇ represents H or a (C₁-C₆) alkyl;
M represents a single bond or a divalent radical having from 1 to 20 carbon atoms;
E represents a charged or ionizable group having 100 atoms at most;
R₁₈, R₁₉, R₂₀, R₂₁ and R₂₂ represent, independently of one another, H or a (C₁-C₆)alkyl.

9. The polymer of claim 1, wherein the matrix is further based on at least one colored monomer of the following general formula (VI): 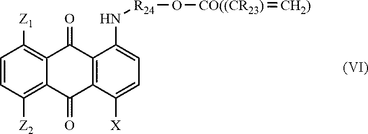
in which
Z₁ and Z₂ represent, independently of one another, H or OR₂₅, R₂₅ representing H or a (C₁-C₆)alkyl;
X represents H or Cl;
R₂₃ represents H or a (C₁-C₆)alkyl; and
R₂₄ represents a group selected from linear or branched (C₁-C₆)alkylene, (C₅-C₃₆)arylene, (C₅-C₃₆)arylene-O-R-₂₆, (C₅-C₃₆)heteroarylene and (C₅-C₃₆)heteroarylene-O-R₂₇, R₂₆ and R₂₇ representing a (C₁-C₆)alkyl or a (C₁-C₆)alkylene.

10. The polymer of claim 1, wherein the matrix is further based on particles visible in magnetic resonance imaging (MRI).

11. The polymer of claim 8, loaded with a drug or with an active substance or with a diagnostic agent, the drug or the active substance.

12. The polymer of claim 8, loaded with macromolecules selected from the group consisting of enzymes, antibodies, cytokines, growth factors, clotting factors, hormones, plasmids, antisense oligonucleotides, siRNA, ribozymes, DNA enzyme, aptamers, anti-inflammatory proteins, bone morphogenetic proteins (BMP), pro-angiogenic factors, vascular endothelial growth factors (VEGF) and TGF-beta, and angiogenesis inhibitors or antityrosine kinases and mixtures thereof.

13. A pharmaceutical composition comprising at least one of the polymer of claim 1, in association with a pharmaceutically acceptable vehicle.

14. A kit comprising the pharmaceutical composition of claim 13, in association with a pharmaceutically acceptable vehicle for a parenteral administration, and at least one means of injection.

15. A kit comprising the pharmaceutical composition of claim 13 and on the other hand at least one contrast agent for imaging by X-ray, by magnetic resonance or by ultrasonography, and optionally at least one means of injection for parenteral administration, the pharmaceutical composition and the at least one contrast agent being packaged separately.

16. A compound with the following general formula (V): 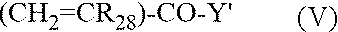
in which
R₂₈ represents H or a (C₁-C₆)alkyl,
Y′ represents (O—R₂₉)t—W′—Ar′, or NH—W′—Ar′, t being an integer between 1 and 10;
R₂₉ represents a group selected from (C₂-C₃₆)alkylene;
W′ represents a single bond, —CONR₃₀—, or —NR₃₁CO—;
Ar′ represents a (C₅-C₃₆)aryl group, said group being substituted with one, two or three atoms of iodine and/or bromine, and optionally substituted with one to four groups selected from (C₁-C₁₀)alkyl, —NR₃₂R₃₃, —NR₃₄COR₃₅, —COOR₃₆, —OR₃₇, —OCOR₃₈, —CONR₃₉R₄₀, —OCONR₄₁R₄₂, —NR₄₃COOR₄₄, NR₄₅CONR₄₆R₄₇, —OCOOR₄₈, and —COR₄₉;
R₃₀ and R₃₁ represent, independently of one another, a hydrogen atom or a (C₁-C₆)alkyl;
R₃₂ to R₄₉ represent, independently of one another, a hydrogen atom, a (C₁-C₁₀)alkyl, said (C₁-C₁₀)alkyl optionally being substituted with 1 to 10 OH groups, or a group —(CH₂—CH₂—O)t′—R″, R″ being a hydrogen atom or a -(C₁-C₆)alkyl and t′ being an integer between 1 and 10.

17. A radiopaque halogenated monomer comprising the compound of general formula (V) of claim 16.

18. The polymer of claim 8, wherein E is selected from the group consisting of —COOH, —COO⁻, —SO₃H, —SO₃⁻, —PO₄H₂, —PO₄H⁻, —PO₄²⁻, —NR₁₈R₁₉, —NR₂₀R₂₁R₂₂⁺.

19. The polymer of claim 9, wherein in the at least one colored monomer of formula (VI), Z₁ and Z₂ represent H, X represents H and R₂₄ represents a group —C₆H₄—O—(CH₂)₂—O or —C(CH₃)₂—CH₂—O.

20. The polymer of claim 11, wherein the drug or the active substance is selected from the group consisting of anti-inflammatory agents, local anesthetics, analgesics, antibiotics, anticancer agents, steroids, antiseptics and a mixture thereof.
